(19) United States
(12) Patent Application Publication
(10) Pub. No.: US 2006/0166992 A1
(43) Pub. Date: Jul. 27, 2006

(54) Heterocyclically substituted imidazotriazines

(75) Inventors: Martin Hendrix, Odenthal (DE); David Bruckner, Essen (DE); Arno Friedl, Bergisch Gladbach (DE); Irene Gerlach, Lorrach (DE); Volker Hinz, Koln (DE); Jorg Keldenich, Wuppertal (DE); Frank Mauler, Odenthal (DE); Ulrich Niewohner, Wermelskirchen (DE); Maria Theresia Niewohner, Wermelskirchen (DE); Dagmar Karthaus, Solingen (DE); Karl-Heinz Schlemmer, Wuppertal (DE); Adrian Tersteeger, Velbert (DE); Ozkan Yalkinoglu, Wuppertal (DE)

Correspondence Address: JEFFREY M. GREENMAN, BAYER PHARMACEUTICALS CORPORATION, 400 MORGAN LANE, WEST HAVEN, CT, 06516, US

(73) Assignee: Bayer Healthcare A G, Leverkusen (DE)

(21) Appl. No.: 10/519,134

(22) PCT Filed: Jun. 25, 2003
(86) PCT No.: PCT/EP03/06662

§ 371 Date: Oct. 7, 2005

(30) Foreign Application Priority Data
Jul. 8, 2002 (DE) 102 30 604.4

(51) Int. Cl. A61K 31/53; C07D 487/04

(52) U.S. Cl. 514/243; 544/184

(57) ABSTRACT

The invention relates to new heterocyclically substituted imidazotriazines, processes for their preparation and their use for the production of medicaments for the treatment and/or prophylaxis of cancer and neurodegenerative disorders, in particular of Parkinson's disease and of schizophrenia.

The invention relates to new heterocyclically substituted imidazotriazines, processes for their preparation and their use for the production of medicaments for the treatment and/or prophylaxis of cancer and neurodegenerative disorders, in particular of Parkinson's disease and of schizophrenia.

The cyclic nucleotides cGMP and cAMP belong to the most important intracellular messenger substances. Phosphodiesterases (PDEs) play a significant role in the regulation of the concentration of cGMP and cAMP. So far, 11 phosphodiesterase isoenzyme groups are known (PDE 1-7: Beavo et al. Mol. Pharmacol. 1994, 399-405; PDE 8-10: Soderling and Beavo Curr. Opin. Cell Biol. 2000, 12, 174-179; PDE 11: Fawcett et al. Proc. Natl. Acad. Sci. U.S.A. 2000, 97, 3702-3707).

PDE 10A hydrolyzes both cAMP and cGMP (Fujishige J. Biol. Chem. 1999, 274, 18438-18445). Transcribed PDE 10A was identified especially in the putamen and caudate nucleus regions of the brain, and in thyroid and testicular tissue. In comparison to normal tissue, the PDE 10A mRNA is moreover strongly expressed in certain tumor tissues, such as, for example, in tissues of breast, liver, colon and lung tumors.

Parkinson's disease is a chronically progressive, neurodegenerative disorder, which is characterized by the loss of dopaminergic neurones of the substantia nigra. The massive disorders of dopaminergic neurotransmission caused thereby lead to a serious malfunction of the movement-controlling extrapyramidal system. The main characteristics of early signs and symptoms of Parkinson's disease are resting tremor, slowing down of movements, muscle stiffness and unstable posture.

The present medications for Parkinson's disease are of purely symptomatic nature, substitution therapy with L-dopa being the most frequently used form of therapy. Neither preventative nor restorative therapies are presently available (Mendis et al., Can. J. Neurol. Sci. 1999, 26, 89-103).

Idiopathic Parkinson's disease is a chronic, progressive neurological disorder, which belongs to a relatively wide classification of neurological diseases which are designated as parkinsonism. It is clinically defined by the occurrence of at least two of the four cardinal symptoms: bradykinesia, resting tremor, muscle stiffness and postural and movement disorders. Pathologically, the idiopathic form of Parkinson's disease is characterized by the loss of pigmented nerve cells, in particular in the area of the substantia nigra of the brain. Idiopathic Parkinson's disease makes up about 75% of all parkinsonism diseases. The other 25% of the cases are designated as atypical parkinsonism and include syndromes such as multiple system atrophy, striatonigral degeneration or vascular parkinsonism.

Schizophrenia is a chronic psychiatric disease which is characterized by psychoses, “negative symptoms” such as apathy and social seclusion, subtle cognitive deficits and lack of understanding of the illness. The etiology and the exact pathophysiology of schizophrenia and related schizoaffective disorders is still not known in detail even today (Kurachi, Psychiatry Clin. Neurosci. 2003, 57, 3-15; Lewis and Levitt, Ann. Rev. Neurosci. 2002, 25, 409-432). In postmortem investigations in the brain of schizophrenic individuals, abnormal cell distributions were found in various regions of the brain and altered brain activation patterns were seen in schizophrenia patients in neuroimaging studies (Goff et al., Med. Clin. N. Am. 2001, 85, 663-689). There are indications that cGMP could be involved in the pathogenesis of psychoses. Thus, Gattaz and coworkers (Gattaz et al., Br. J. Psychiatry 1983, 142, 288-291) reported that the levels of cGMP in the cerebrospinal fluid of schizophrenic patients are altered. Moreover, it was shown that the administration of the classic antipsychotic haloperidol increases the cGMP content of the cerebrospinal fluid (Gattaz et al., Biol. Psychiatry 1984, 19, 1229-35).

Although the details of the neuroanatomic basis of schizophrenic disorders are still the subject of medical research, it was possible to show that, inter alia, the basal ganglia play an important role in these diseases (e.g. Shenton et al., Schizophr. Res. 2001, 49, 1-52).

The synthesis of 4-amino-2,5-diphenyl-7-methylthio-imidazo[5,1-f]-[1,2,4]triazines is known from Synthesis 1989, 843-847.

In U.S. Pat. No. 3,941,785,2-amino-imidazo[5,1-f]-[1,2,4]triazines are described as PDE inhibitors having spasmolytic action for the treatment of asthma, bronchitis, chronic heart failure and skin diseases.

EP-A 1 250 923 describes the use of selective PDE10 inhibitors, such as, for example, papaverine, for the treatment of diseases of the central nervous system, such as, for example, Parkinson's disease.

The present invention relates to compounds of the formula 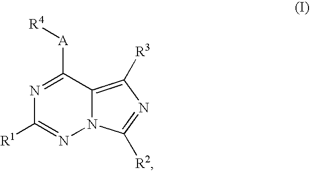
in which
R¹ denotes 5- to 10-membered heteroaryl, which can be substituted by up to 3 substituents selected independently of one another from the group consisting of oxo, halogen, carbamoyl, cyano, hydroxyl, (C₁-C₆-alkyl)carbonyl, trifluoromethyl, trifluoromethoxy, nitro, C₁-C₆-alkyl, C₁-C₆-alkoxy and —NR⁵R⁶,
   where
   R⁵ and R⁶ independently of one another denote for C₁-C₆-alkyl or
   R⁵ and R⁶, together with the nitrogen atom to which they are bonded, denote a 5 to 8-membered heterocycle which is optionally substituted by C₁-C₆-alkyl or C₁-C₆-hydroxyalkyl,
R² denotes C₁-C₆-alkyl or C₃-C₄-cycloalkyl,
R³ denotes methyl,
A denotes oxygen or NH, and
R⁴ denotes C₆-C₁₀-aryl, which can be substituted by up to 3 substituents selected independently of one another from the group consisting of halogen, formyl, carboxyl, carbamoyl, cyano, hydroxyl, trifluoromethyl, trifluoromethoxy, nitro, C₁-C₆-alkyl, C₁-C₆-alkoxy, 1,3-dioxa-propane-1,3-diyl, C₁-C₆-alkylthio and —NR⁷R⁸,
   in which
   R⁷ and R⁸ independently of one another denote hydrogen, C₁-C₆-alkyl or C₁-C₆-alkylcarbonyl,
      and their salts, solvates and/or solvates of the salts.

Depending on their structure, the compounds according to the invention can exist in stereoisomeric forms (enantiomers, diastereomers). The invention therefore relates to the enantiomers or diastereomers and their respective mixtures. The stereoisomerically uniform constituents can be isolated in a known manner from such mixtures of enantiomers and/or diastereomers.

Salts which are preferred in the context of the invention are physiologically acceptable salts of the compounds according to the invention.

Physiologically acceptable salts of the compounds (I) include acid addition salts of mineral acids, carboxylic acids and sulfonic acids, e.g. the salts of hydrochloric acid, hydrobromic acid, sulfuric acid, phosphoric acid, methanesulfonic acid, ethanesulfonic acid, toluenesulfonic acid, benzenesulfonic acid, naphthalenedisulfonic acid, acetic acid, propionic acid, lactic acid, tartaric acid, malic acid, citric acid, fumaric acid, maleic acid and benzoic acid.

Physiologically acceptable salts of the compounds (I) also include salts of customary bases, such as, by way of example and preferably, alkali metals salts (e.g. sodium and potassium salts), alkaline earth metal salts (e.g. calcium and magnesium salts) and ammonium salts, derived from ammonia or organic amines having 1 to 16 C atoms, such as, by way of example and preferably, ethylamine, diethylamine, triethylamine, ethyldiisopropylamine, monoethanolamine, diethanolamine, triethanolamine, dicyclohexylamine, dimethylaminoethanol, procaine, dibenzylamine, N-methylmorpholine, dehydroabietylamine, arginine, lysine, ethylenediamine and methylpiperidine.

Solvates in the context of the invention are designated as those forms of the compounds which in the solid or liquid state form a complex by coordination with solvent molecules. Hydrates are a special form of the solvates, in which the coordination takes place with water.

In the context of the present invention the substituents, if not stated otherwise, have the following meaning:

C₁-C₆-Alkoxy represents a straight-chain or branched alkoxy radical having 1 to 6, preferably 1 to 4, particularly preferably having 1 to 3 carbon atoms. Nonlimiting examples include methoxy, ethoxy, n-propoxy, isopropoxy, tert-butoxy, n-pentoxy and n-hexoxy.

C₁-C₆-Alkyl represents a straight-chain or branched alkyl radical having 1 to 6, preferably 1 to 4, particularly preferably 1 to 3 carbon atoms. Nonlimiting examples include methyl, ethyl, n-propyl, isopropyl, tert-butyl, n-pentyl and n-hexyl.

(C₁-C₆-Alkyl)carbonyl represents a straight-chain or branched alkylcarbonyl radical having 1 to 6, preferably 1 to 4, particularly preferably 1 to 3 carbon atoms. Nonlimiting examples include acetyl, ethylcarbonyl, propylcarbonyl, isopropylcarbonyl, butylcarbonyl, isobutylcarbonyl, pentylcarbonyl and hexylcarbonyl.

C₁-C₆-Alkylthio represents a straight-chain or branched alkylthio radical having 1 to 6, preferably 1 to 4, particularly preferably 1 to 3 carbon atoms. Nonlimiting examples include methylthio, ethylthio, n-propylthio, isopropylthio, tert-butylthio, n-pentylthio and n-hexylthio.

C₆-C₁₀-Aryl represents an aromatic radical having 6 to 10 carbon atoms. Nonlimiting examples include phenyl and naphthyl.

Halogen represents fluorine, chlorine, bromine and iodine. Fluorine, chlorine and bromine are preferred, particularly preferably fluorine and chlorine.

5- to 10-membered heteroaryl represents an aromatic, mono- or bicyclic radical having 5 to 10 ring atoms and up to 5 heteroatoms selected from the group consisting of S, O and/or N. 5- to 6-membered heteroaryls having up to 4 heteroatoms are preferred. The heteroaryl radical can be bonded via a carbon or heteroatom. Nonlimiting examples include thienyl, furyl, pyrrolyl, thiazolyl, oxazolyl, imidazolyl, pyridyl, pyrimidyl, pyridazinyl, indolyl, indazolyl, benzofuranyl, benzothiophenyl, quinolinyl, isoquinolinyl.

5 to 8-membered heterocycle represents a mono- or polycyclic, heterocyclic radical having 5 to 8 ring atoms and up to 3, preferably 2, heteroatoms or hetero groups from the group consisting of N, O, S, SO, SO₂, where at least one of the heteroatoms or hetero groups is a nitrogen atom. 5- to 7-membered heterocyclyl is preferred. Mono- or bicyclic heterocyclyl is preferred. Monocyclic heterocyclyl is particularly preferred. As heteroatoms, O, N and S are preferred. The heterocyclyl radicals can be saturated or partially unsaturated. Saturated heterocyclyl radicals are preferred. 5- to 7-membered, monocyclic saturated heterocyclyl having up to two heteroatoms from the group consisting of O, N and S is particularly preferred. Nonlimiting examples include pyrrolinyl, piperidinyl, morpholinyl, perhydroazepinyl.

C₁-C₄-Cycloalkyl represents monocyclic cycloalkyl, for example cyclopropyl and cyclobutyl.

C₁-C₆-Hydroxyalkyl represents a straight-chain or branched hydroxyalkyl radical having 1 to 6, preferably 1 to 4, particularly preferably 1 to 3 carbon atoms. Nonlimiting examples include hydroxymethyl, 1- or 2-hydroxyethyl, 1-, 2- or 3-n-hydroxypropyl, 1- or 2-hydroxyisopropyl, 1-hydroxy-tert-butyl, 1-, 2-, 3-, 4- or 5-n-hydroxypentyl and 1-, 2-, 3-, 4-, 5- or 6-n-hydroxyhexyl.

If radicals in the compounds according to the invention are optionally substituted, if not specified otherwise, a substitution by up to three identical or different substituents is preferred.

The compounds according to the invention can also be present as tautomers, as is shown by way of example below for A=NH:

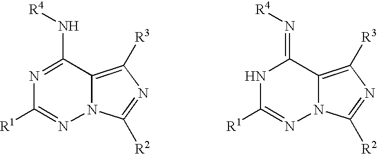

A further embodiment of the invention relates to compounds of the formula (1),

in which

R¹ denotes 5- to 10-membered heteroaryl, which can be substituted by up to 3 substituents selected independently of one another from the group consisting of oxo, C₁-C₆-alkyl, C₁-C₆-alkoxy and —NR⁵R⁶,
   where
   R⁵ and R⁶ independently of one another denote C₁-C₆-alkyl or
   R⁵ and R⁶, together with the nitrogen atom to which they are bonded, form a 5 to 8-membered heterocycle, which is optionally substituted by C₁-C₆-alkyl or C₁-C₆-hydroxyalkyl,
R² denotes C₁-C₆-alkyl,
R³ denotes methyl,
A denotes oxygen or NH, and
R⁴ denotes phenyl, which can be substituted by up to 3 substituents selected independently of one another from the group consisting of halogen, C₁-C₆-alkyl and C₁-C₆-alkoxy,
   and their salts, solvates and/or solvates of the salts.

A further embodiment of the invention relates to compounds of the formula (I),

in which

R¹ denotes 5- to 6-membered heteroaryl, which can be substituted by up to 3 substituents selected independently of one another from the group consisting of oxo, C₁-C₆-alkyl, C₁-C₆-alkoxy and —NR⁵R⁶,
   where R⁵ and R⁶ independently of one another denote C₁-C₆-alkyl or
   R⁵ and R⁶, together with the nitrogen atom to which they are bonded, form a 5 to 8-membered heterocycle, which is optionally substituted by C₁-C₆-alkyl or C₁-C₆-hydroxyalkyl, and
R², R³, R⁴ and A have the abovementioned meanings
   and their salts, solvates and/or solvates of the salts.

A further embodiment of the invention relates to compounds of the formula (I),

in which

R¹ represents thienyl, furyl, thiazolyl or pyridyl, which in each case can be substituted by up to 2 substituents selected independently of one another from the group consisting of oxo, C₁-C₆-alkyl, C₁-C₆-alkoxy and —NR⁵R⁶,
   where R⁵ and R⁶ independently of one another denote C₁-C₆-alkyl or
   R⁵ and R⁶, together with the nitrogen atom to which they are bonded, form a 5 to 8-membered heterocycle, which is optionally substituted by C₁-C₆-alkyl or C₁-C₆-hydroxyalkyl, and
R², R³, R⁴ and A have the abovementioned meanings
   and their salts, solvates and/or solvates of the salts.

A further embodiment of the invention relates to compounds of the formula (I),

in which

R¹ denotes meta-pyridyl, which can be substituted by up to 2 substituents selected independently of one another from the group consisting of oxo, C₁-C₆-alkyl, C₁-C₆-alkoxy and —NR⁵R⁶,
   where R⁵ and R⁶ independently of one another denote C₁-C₆-alkyl or
   R⁵ and R⁶, together with the nitrogen atom to which they are bonded, form a 5 to 8-membered heterocycle, which is optionally substituted by C₁-C₆-alkyl or C₁-C₆-hydroxyalkyl, and
R², R³, R⁴ and A have the abovementioned meanings
   and their salts, solvates and/or solvates of the salts.

A further embodiment of the invention relates to compounds of the formula (I),

in which

R² denotes C₁-C₆-alkyl and
R¹, R³, R⁴ and A have the abovementioned meanings, and their salts, solvates and solvates of the salts.

A further embodiment of the invention relates to compounds of the formula (I),

in which

R⁴ denotes phenyl, which can be substituted by up to 3 C₁-C₆-alkoxy radicals, and
R¹, R², R³ and A have the abovementioned meanings
   and their salts, solvates and/or solvates of the salts.

A further embodiment of the invention relates to compounds of the formula (I),

in which

R⁴ denotes 3,4,5-trimethoxyphenyl and
R¹, R², R³ and A have the abovementioned meanings
   and their salts, solvates and/or solvates of the salts.

The invention furthermore relates to processes for the preparation of the compounds according to the invention, according to which compounds of the formula 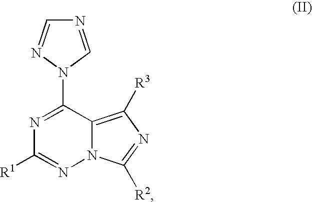
in which R¹, R² and R³ have the meanings indicated above,
are reacted with compounds of the formula 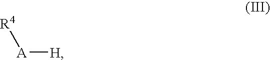
in which R⁴ and A have the meanings indicated above,
to give compounds of the formula (I) and these are optionally reacted with the appropriate (i) solvents and/or (ii) bases or acids to give their solvates, salts and/or solvates of the salts.

The reaction is carried out in inert solvents or without solvents in the melt, if appropriate in the presence of base and/or auxiliary reagents, preferably in a temperature range from 20° C. up to reflux of the solvents at normal pressure.

Inert solvents are, for example, halogenohydrocarbons such as methylene chloride, trichloromethane, tetrachloromethane, trichloroethane, tetrachloroethane, 1,2-dichloroethane or trichloroethylene, ethers such as diethyl ether, methyl tert-butyl ether, dioxane, tetrahydrofuran, 1,2-dimethoxyethane, or diethylene glycol dimethyl ether, hydrocarbons such as benzene, xylene, toluene, hexane, cyclohexane or petroleum fractions, nitroalkanes such as nitromethane, carboxylic acid esters such as ethyl acetate, carboxamides such as dimethylformamide, dimethylacetamide, alkyl sulfoxides such as dimethyl sulfoxide, alkylnitriles such as acetonitrile or heteroaromatics such as pyridine, preferably pyridine, glycol dimethyl ether, diethylene glycol dimethyl ether, tetrahydrofuran, dioxane or dimethyl sulfoxide; a reaction without solvent in the melt is also preferred.

Bases are, for example, alkali metal hydroxides such as sodium hydroxide or potassium hydroxide, alkali metal carbonates such as cesium carbonate, sodium carbonate or potassium carbonate, alkali metal alkoxides such as sodium methoxide or potassium methoxide, sodium ethoxide or potassium ethoxide or potassium tert-butoxide, amides such as sodium amide, lithium bis-(trimethylsilyl)amide, lithium diisopropylamide, organometallic compounds such as butyllithium or phenyllithium, alkali metal hydrides such as sodium hydride, organic amines such as DBU, triethylamine or diisopropylethylamine, preferably sodium hydride, triethylamine, potassium tert-butoxide or DBU.

Auxiliary reagents are, for example, potassium fluoride or dimethylaminopyridine or/and crown ethers, preferably 15-crown-5, 18-crown-8 or 12-crown-4.

The compounds (III) are known or can be synthesized from the corresponding starting materials analogously to known processes.

For the preparation of the compounds (II), compounds of the formula 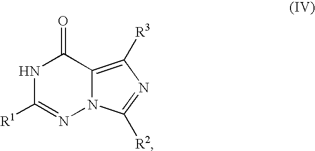
in which
R¹, R² and R³ have the meanings indicated above,
   can be reacted with 1,2,4-triazole in the presence of a chlorinating agent, preferably phosphorus oxychloride, phosphorus pentachloride, sulfuryl chloride and/or thionyl chloride.

The reaction is in general carried out in inert solvents, if appropriate in the presence of a base, preferably in a temperature range from −20° C. to 20° C. at normal pressure (cf., for example, Knutsen et al., J. Chem. Soc., Perkin Trans 1, 1985, 621-630; A. Kraszewski, J. Stawinski, Tetrahedron Lett. 1980, 21, 2935).

Preferred inert solvents are pyridine, trichloromethane, diethylphenylamine, dioxane or acetonitrile.

Preferred bases are triethylamine, pyridine or diethylphenylamine.

For the preparation of the compounds (IV), compounds of the formula 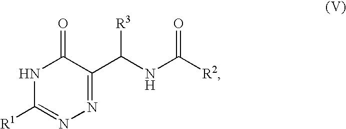
in which
R¹, R² and R³ have the meanings indicated above,
   can be reacted with suitable dehydrating reagents (e.g. Lewis acids), preferably phosphorus oxychloride, phosphorus pentoxide, polyphosphoric acid or methylsulfonyl chloride.

The reaction can be carried out in inert solvents, preferably in a temperature range from 40 to 80° C. at normal pressure (cf., for example, Charles et al. J. Chem. Soc., Perkin Trans 1, 1980, 1139).

1,2-Dichloroethane is preferred as an inert solvent.

For the preparation of the compounds (V), compounds of the formula 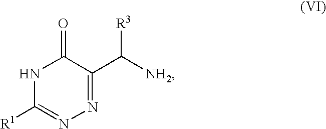
or their salts, e.g. hydrochloride salts, in which R¹ and R³ have the meanings indicated above,
can be reacted with compounds of the formula 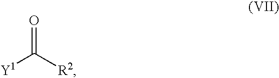
in which
R² has the meaning indicated above and
Y¹ represents halogen, preferably bromine or chlorine, or hydroxyl.

If Y¹ represents halogen, the reaction can be carried out in inert solvents, if appropriate in the presence of a base, preferably in a temperature range from 0° C. to 50° C. at normal pressure.

Tetrahydrofuran or methylene chloride are preferred as inert solvents.

Triethylamine is preferred as a base.

If Y¹ represents hydroxyl, the reaction can be carried out in inert solvents, if appropriate in the presence of a base and/or condensing agents, preferably in a temperature range from 20° C. to 50° C. at normal pressure.

Condensing agents are, for example, carbodiimides such as, for example, N,N′-diethyl-, N,N,′-dipropyl-, N,N′-diisopropyl-, N,N′-dicyclohexylcarbodiimide, N-(3-dimethylaminoisopropyl)-N′-ethylcarbodiimide hydrochloride (EDC), N-cyclohexylcarbodiimide-N′-propyloxymethyl-polystyrene (PS carbodiimide), carbonyl compounds such as carbonyldiimidazole, 1,2-oxazolium compounds such as 2-ethyl-5-phenyl-1,2-oxazolium 3-sulfate or 2-tert-butyl-5-methyl-isoxazolium perchlorate, acylamino compounds such as 2-ethoxy-1-ethoxycarbonyl-1,2-dihydroquinoline, propanephosphonic anhydride or isobutyl chloroformate or bis-(2-oxo-3-oxazolidinyl)-phosphoryl chloride or benzotriazolyloxy-tri(dimethylamino)phosphonium hexafluorophosphate or O-(benzotriazol-1-yl)-N,N,N′,N′-tetra-methyluronium hexafluorophosphate (HBTU) or 2-(2-oxo-1-(2H)-pyridyl)-1,1,3,3-tetramethyluronium tetrafluoroborate (TPTU) or O-(7-azabenzotriazol-1-yl)-N,N,N′,N′-tetramethyluronium hexafluorophosphate (HATU) or 1-hydroxybenzotriazole (HOBt) or benzotriazol-1-yloxytris(dimethylamino)phosphonium hexafluorophosphate (BOP) or mixtures of these compounds.

Bases are, for example, alkali metal carbonates, e.g. sodium or potassium carbonate or sodium or potassium hydrogencarbonate, or organic bases such as trialkylamines, e.g. triethylamine, N-methylmorpholine, N-methylpiperidine, 4-dimethylaminopyridine or diisopropylethylamine.

The combination of N-(3-dimethylaminoisopropyl)-N′-ethylcarbodiimide hydrochloride (EDC) and 1-hydroxybenztriazole (HOBt), and the combination of benzotriazol-1-yloxytris(dimethylamino)phosphonium hexafluorophosphate (BOP) and triethylamine are particularly preferred.

The compounds (VII) are known or can be synthesized from the corresponding starting materials analogously to known processes.

For the preparation of the compounds (VI), compounds of the formula 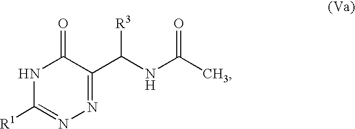
in which
R¹ and R³ have the meanings indicated above,
   can be reacted with an acid.

The reaction can be carried out in inert solvents, preferably in a temperature range from 20° C. to 100° C. at normal pressure.

In addition to the inert solvents already mentioned, alcohols such as methanol, ethanol, n-propanol, iso-propanol, n-butanol or tert-butanol, preferably methanol or ethanol, can be used in this reaction.

Acids are, for example, organic acids such as acetic acid and trifluoroacetic acid or inorganic acids such as sulfuric acid, hydrogen chloride and hydrogen bromide or their mixtures, if appropriate with addition of water; hydrogen chloride or hydrogen chloride/water is particularly preferred.

In an alternative process for the preparation of the compounds (V), compounds of the formula 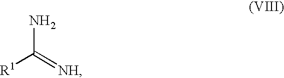
or their salts, e.g. hydrochloride or hydrobromide salts,
in which
R¹ has the meaning indicated above,
   can be reacted in the first stage with hydrazine and the resulting reaction product can be reacted in a second stage with compounds of the formula 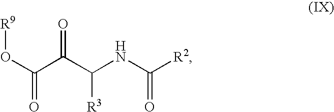
   in which
R² and R³ have the meanings indicated above and
R⁹ represents (C₁-C₄)-alkyl, preferably methyl or ethyl.

The reaction of the first stage can be carried out in inert solvents, preferably in a temperature range from −10° C. to 50° C. at normal pressure (cf., for example, K. M. Doyle, F. Kurzer, Synthesis 1974, 583).

The reaction of the second stage can be carried out in inert solvents, preferably in a temperature range from 20 to 120° C. at normal pressure.

Inert solvents are, for example, alcohols such as methanol, ethanol, n-propanol, iso-propanol, n-butanol or tert-butanol, carboxamides such as dimethylformamide or alkyl sulfoxides such as dimethyl sulfoxide; methanol or ethanol are preferred.

The compounds (Va) can be prepared using compounds (VIII) and compounds (IX),

in which R² represents methyl, under the same conditions as the compounds (V).

For the preparation of the compounds (VIII), compounds of the formula
R¹—Y² (X),
in which
R¹ has the meaning indicated above and
Y² represents alkoxycarbonyl, preferably methoxycarbonyl or ethoxycarbonyl, or cyano,
   can be reacted with trimethylaluminum.

Preferably, the reaction can be carried out in straight-chain hydrocarbons, e.g. hexane, as an inert solvent and with addition of ammonium salts such as ammonium chloride.

The reaction can be carried out in inert solvents, preferably in a temperature range from initially at −20° C. and subsequently at 20° C. to 80° C. at normal pressure (cf., for example, for cyano: R. S. Garigipati, Tetrahedron Lett. 1990, 31, 1969-1972; for alkoxycarbonyl: H. Gielen, C. Alonso-Alija, M. Hendrix, U. Niewöhner, D. Schauss, Tetrahedron Lett. 2002, 43, 419-421).

As an inert solvent, toluene is preferred.

If Y² represents cyano, in an alternative process the reaction can be carried out using ammonium bromide or chloride and gaseous ammonia at 140° C. to 150° C. in an autoclave or using lithium bis(trimethylsilyl)amine and hydrogen chloride in diethyl ether (cf. R. T. Boeré, et al., J. Organomet. Chem. 1987, 331, 161-167).

The compounds (X) are known or can be synthesized from the corresponding starting materials analogously to known processes.

Instead of the compounds (VIII), compounds of the formula 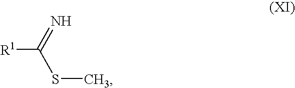
in which
R¹ has the meaning indicated above,
   can also be employed. The compounds (XI) can be prepared according to K. M. Doyle, F. Kurzer, Synthesis 1974, 583.

For the preparation of the compounds (IX), compounds of the formula 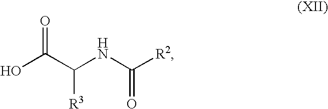
in which
R² and R³ have the meanings indicated above,
   can be reacted with compounds of the formula 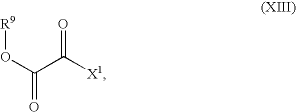
   in which
R⁹ has the meaning indicated above and
X¹ represents halogen, preferably chlorine or bromine.

The reaction can be carried out in inert solvents, if appropriate in the presence of base and/or of a catalyst such as dimethylaminopyridine, preferably in a temperature range from 20 to 80° C. at normal pressure (cf., for example, Charles, J. Chem. Soc., Perkin Trans. 1, 1980, 1139).

Preferred inert solvents are tetrahydrofuran or diethyl ether.

The compounds (XIII) are known or can be synthesized from the corresponding starting materials analogously to known processes.

For the preparation of the compounds (XII), compounds of the formula 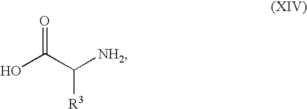
in which
R³ has the meaning indicated above,
   can be reacted with compounds of the formula 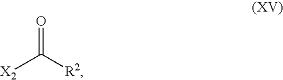
   in which
R² has the meaning indicated above and
X² represents halogen, preferably chlorine or bromine.

The reaction can be carried out in inert solvents, if appropriate in the presence of a base and trimethylsilyl chloride, preferably in a temperature range from −10 to 60° C. at normal pressure.

A preferred inert solvent is methylene chloride.

Bases are, for example, alkali metal hydroxides such as sodium hydroxide or potassium hydroxide, optionally as a mixture with water, alkali metal carbonates such as cesium carbonate, sodium carbonate or potassium carbonate, alkali metal alkoxides such as potassium tert-butoxide, or amides such as sodium amide, lithium bis-(trimethylsilyl)amide, lithium diisopropylamide, organic amines such as DBU, triethylamine, pyridine, piperidine or diisopropylethylamine, preferably triethylamine, sodium hydroxide or potassium hydroxide as a mixture with water.

The compounds (XIV) and (XV) are known or can be synthesized from the corresponding starting materials analogously to known processes.

For the syntheses of intermediates for the preparation of the compounds (I), the methods described in WO 99/24433 and EP-A-1 092 719 are optionally also used.

Functional groups are optionally protected during the syntheses by protective groups, which can subsequently be removed again (cf., for example, T. W. Greene, P. Wuts, “Protective Groups in Organic Synthesis”, 2nd Ed., Wiley; New York, 1991).

The processes described above can be illustrated by way of example by the following reaction schemes:

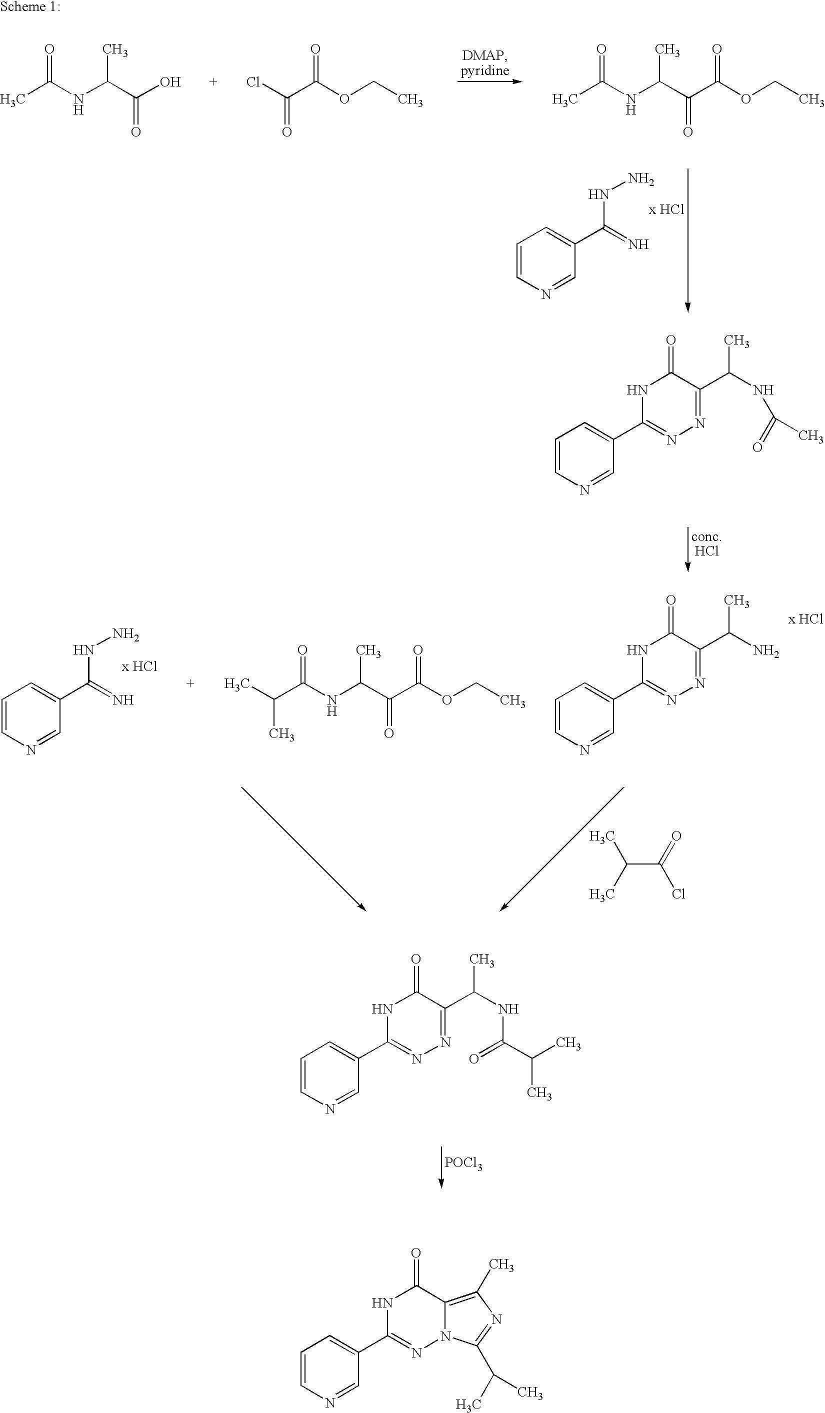

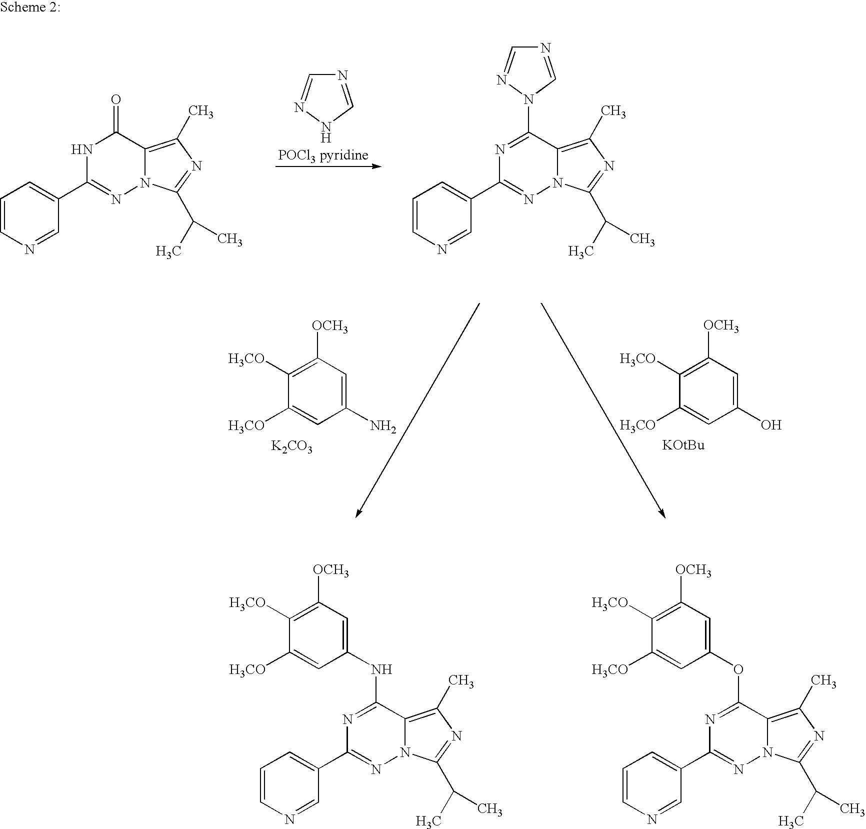

The compounds according to the invention show an unforeseeable, valuable spectrum of pharmacological action. They are distinguished as PDE 10A inhibitors.

It was possible for the first time to show selective PDE 10A inhibition in animal models which makes a connection between PDE 10A inhibitors and Parkinson's disease.

On account of their pharmacological properties, the compounds according to the invention can be used on their own or in combination with other medicaments for the treatment and/or prevention of Parkinson's disease, in particular of idiopathic Parkinson's disease, and of cancers, in particular of tumors, and for the treatment of schizophrenia.

Idiopathic Parkinson's disease is a chronic, progressive neurological disorder, which belongs to a relatively wide classification of neurological diseases which are designated as parkinsonism. It is clinically defined by the occurrence of at least two of the four cardinal symptoms: bradykinesia, resting tremor, muscle stiffness and postural and movement disorders. Pathologically, the idiopathic form of Parkinson's disease is characterized by the loss of pigmented nerve cells, in particular in the area of the substantia nigra of the brain. Idiopathic Parkinson's disease makes up about 75% of all parkinsonism diseases. The other 25% of the cases are designated as atypical parkinsonism and include syndromes such as multiple system atrophy, striatonigral degeneration or vascular parkinsonism.

In the context of the present invention, the definition of tumors includes both benign and malignant tumors and thus, for example, also benign neoplasias, dysplasias, hyperplasias, and neoplasias with metastasis formation. Further examples of tumors are carcinomas, sarcomas, carcinosarcomas, tumors of the blood-forming organs, tumors of the nervous tissue, for example of the brain, or tumors of skin cells. In tumor formation, uncontrolled or inadequately controlled cell division occurs. The tumor can be locally restricted, but it can also infiltrate the surrounding tissue and then get lodged by the lymphatic system or by the bloodstream in a new location. There are thus primary and secondary tumors. Primary tumors are originally formed in the organ in which they are found. Secondary tumors have been lodged in another organ by metastasis formation and then spread in their new location.

An abnormal function of the basal ganglia is not only relevant for psychoses, schizophrenia and related schizoaffective disorders, but also plays a role for other neuropsychiatric changes such as depression (Kapur, Biol. Psychiatr. 1992, 32, 1-17; Lafer, et al., Psychiatr. Clin. North. Am. 1997, 20, 855-896) and anxiety disorders (Jetty, et al., Psychiatr. Clin. North. Am. 2001, 24, 75-97).

Furthermore, the compounds according to the invention are suitable for the treatment of further diseases which can be treated by influencing the cGMP level and/or the cAMP level, such as dementia, stroke, craniocerebral trauma, Alzheimer's disease, dementia with frontal lobe degeneration, Lewy body dementia, vascular dementia, attention deficit syndrome, attention and concentration disorders, affective disorders, psychoses, neuroses, mania or manic depressive disorders, Pick's disease, pain and epilepsy.

The in vitro action of the compounds according to the invention can be shown using the following biological assays:

In Vitro Enzyme Inhibition Tests:

Inhibition of PDE 10A

PDE 10A (WO 01/29 199, FIG. 1A) is expressed recombinantly in full length in Sf9 insect cells (Invitrogen, Carlsbad, Calif.) with the aid of the Bac-to-Bac™ Baculovirus expression system from Life Technologies (Gaithersburg, Md.). 48 h after infection, the cells are harvested and suspended in 20 ml (per 11 of culture) of lysis buffer (50 mM tris HCl, pH 7.4, 50 mM NaCl, 1 mM MgCl₂, 1.5 mM EDTA, 10% glycerol plus 20 μl of Protease Inhibitor Cocktail Set II [CalBiochem, La Jolla, Calif. USA]). The cells are treated with ultrasound at 4° C. for 1 minute and subsequently centrifuged at 10 000 rpm for 30 minutes at 4° C. The supernatant (PDE 10A preparation) was collected and stored at −20° C.

The test substances are dissolved in 100% DMSO for the determination of their in vitro action on PDE 10A and serially diluted. Typically, dilution series from 200 μM to 1.6 μM are prepared (resulting final concentrations in the test: 4 μM to 0.032 μM). 2 μl of the diluted substance solutions in each case are introduced into the hollows of microtiter plates (Isoplate; Wallac Inc., Atlanta, Ga.). Subsequently, 50 μl of a dilution of the PDE 10A preparation described above are added. The dilution of the PDE 10A preparation is chosen such that during the later incubation less than 70% of the substrate is reacted (typical dilution: 1:10 000; dilution buffer: 50 mM tris/HCl pH 7.5, 8.3 mM MgCl₂, 1.7 mM EDTA, 0.2% BSA). The substrate, [5′,8-³H] adenosine 3′,5′-cyclic phosphate (1 μCi/μl; Amersham Pharmacia Biotech., Piscataway, N.J.) is diluted 1:2000 with assay buffer (50 mM tris/HCl pH 7.5, 8.3 mM MgCl₂, 1.7 mM EDTA) to a concentration of 0.0005 μCi/μl. The enzyme reaction is finally started by addition of 50 μl (0.025 μCi) of the diluted substrate. The test batches are incubated for 60 min at 20° C. and the reaction is stopped by addition of 25 μl of a suspension containing 18 mg/ml of Yttrium Scintillation Proximity Beads (Amersham Pharmacia Biotech., Piscataway, N.J.). The microtiter plates are sealed using a film and allowed to stand for 60 min at 20° C. Subsequently, the plates are measured for 30 s per hollow in a Microbeta scintillation counter (Wallac Inc., Atlanta, Ga.). IC₅₀ values are determined by means of graphic plotting of the substance concentration against the percentage inhibition.

The PDE 10A-inhibiting action of the compounds according to the invention may be shown by the following examples:

| Example | IC₅₀ [nM] |
|---|---|
| 9 | 38 |
| 10 | 8 |
| 12 | 93 |
| 14 | 150 |
| 16 | 30 |

Inhibition of the PDEs 1-5, 7-9 and 11

Recombinant PDE 1C (GenBank/EMBL accession number: NM_{—}005020, Loughney et al. J. Biol. Chem. 1996 271, 796-806), PDE 2A (GenBank/EMBL accession number: NM_{—}002599, Rosman et al. Gene 1997 191, 89-95), PDE3B (GenBank/EMBL accession number: NM_{—}000922, Miki et al. Genomics 1996 36, 476-485), PDE 4B (GenBank/EMBL accession number: NM_{—}002600, Obernolte et al. Gene. 1993 129, 239-247), PDE 5A (GenBank/EMBL accession number: NM_{—}001083, Loughney et al. Gene 1998 216, 139-147), PDE 7B (GenBank/EMBL accession number: NM_{—}018945, Hetman et al. Proc. Natl. Acad. Sci. U.S.A. 2000 97, 472-476), PDE 8A (GenBank/EMBL accession number: AF-056490, Fisher et al. Biochem. Biophys. Res. Commun. 1998 246, 570-577), PDE 9A (GenBank/EMBL accession number: NM_{—}002606, Fisher et al. J. Biol. Chem. 1998 273, 15559-15564), PDE 11A (GenBank/EMBL accession number: NM_{—}016953, Fawcett et al. Proc. Natl. Acad. Sci 2000 97, 3702-3707) were expressed in Sf9 cells with the aid of the pFASTBAC Baculovirus expression system (GibcoBRL).

The in vitro action of test substances on recombinant PDE 3B, PDE 4B, PDE 7B, PDE 8A and PDE 11A is determined according to the test protocol described above for PDE 10A. For the determination of a corresponding action on recombinant PDE 1C, PDE 2A, PDE5A and PDE 9A, the protocol is adapted as follows: In the case of PDE 1C, calmodulin (10⁻⁷ M) and CaCl₂ (3 mM) are additionally added to the reaction batch. PDE 2A is stimulated in the test by addition of cGMP (1 μM) and tested using a BSA concentration of 0.01%. For PDE 5A and PDE 9A, [8-³H] cGMP (Amersham Pharmacia Biotech., Piscataway, N.J.) is employed as a substrate.

The suitability of the compounds according to the invention for the treatment of Parkinson's disease can be shown in the following animal models:

Haloperidol Catalepsy of Rats

The neuroleptic haloperidol is a high-affinity antagonist on the dopamine D2 receptor. In humans and animals, the administration of a relatively high dose of haloperidol causes a transient blockade of dopaminergic neurotransmission. This blockade leads to a disorder of the extrapyramidal motor functions, “catalepsy”, in which a given posture is retained for longer than normal. The catalepsy induced in animals by neuroleptics is generally regarded as a model for the hypokinesia and rigidity in Parkinson's patients (Elliott et al., J Neural Transm [P-D Sect] 1990; 2:79-89). The time which an animal needs in order to change a given position is used as an index for the degree of catalepsy (Sanberg et al., Behav. Neurosci. 1988; 102:748-59).

In the catalepsy experiments, male rats were divided at random into groups, to which either vehicle or different doses of the compounds to be tested are administered. Each rat receives an intraperitoneal injection of 1.5 mg/kg of haloperidol. The cataleptic behavior of the animals is recorded 120 min after the administration of haloperidol. The compounds to be tested are administered to the rats at such a time interval before the catalepsy test that at the time of the behavior test the maximum plasma concentration is achieved.

For the measurement of the cataleptic behavior, the animal is placed with both forepaws on a block of wood of 9×5.5×5.5 cm height×width×depth. The time which an animal needs in order to take both paws off the block of wood is recorded as the duration of catalepsy. After 180 sec, the animals are taken from the block.

6-Hydroxydopamine (6-OH-DA) Lesion in Rats

The degeneration of the dopaminergic nigrostriatal and striatopallidal neurotransmission is the main sign of Parkinson's disease. The syndrome of Parkinson's disease can be simulated to large parts in an animal model in which the neurotoxin 6-OH-DA is injected intracerebrally into rats.

For the experiments described, male rats (Harlan Winkelmann, Germany; weight at the start of the experiment: 180-200 g) were kept under controlled conditions (atmospheric humidity, temperature) and a 12 hour light-dark cycle. The animals—provided they are not in an experiment—have free access to water and food.

On the operation day, pargyline (Sigma, St. Louis, Mo., USA; 50 mg/kg i.p.) and desmethylimipramine hydrochloride (Sigma; 25 mg/kg i.p.) are administered to the animals 30 minutes before the lesion in order to suppress the metabolism of 6-hydroxydopamine, or in order to prevent the uptake of 6-hydroxydopamine into noradrenergic structures. After initiating the anesthesia by means of sodium pentobarbital (50 mg/kg i.p.), the experimental animals are fixed in a stereotactic frame. The lesion to the nigrostriatal neurotransmission is carried out by means of a unilateral, single injection of 8 μg of 6-OH-DA hydrobromide (Sigma, St. Louis, Mo., USA), dissolved in 4 μl of a 0.01% strength ascorbic acid-saline solution. The solution is injected slowly (1 μl/min). The coordinates of the injection according to König and Klippel are: 2.4 mm anterior, 1.49 mm lateral, 2.7 mm ventral. After the injection the injection needle was left in situ for another 5 minutes in order to facilitate the diffusion of the neurotoxin.

After the operation, the animals are put onto a warm plate and after waking up under surveillance they are transferred to their cages again, where they received food and water ad libidum.

In the drug group, the animals are treated with substance one day after the operation up to the end of the experiment 28 days after the operation.

Such 6-OHDA-damaged animals are divided into various treatment groups, which receive either vehicle or various doses of the compound to be investigated. For comparison purposes, a group of sham-damaged animals (instead of 6-OHDA 0.9% strength sodium chloride solution in water is injected) is additionally included.

The motor failures resulting from the lesion are quantified using the following tests, as described in the respective literature:

a) Staircase Test (Forepaws Coordination Test):

Barnéoud et al: Effects of complete and partial lesions of the dopaminergic mesotelencephalic system on skilled forelimb use in the rat. Neuroscience 1995, 67, 837-848.

b) Accelerating Rotarod Test (Balancing Test):

Spooren et al.: Effects of the prototypical mGlu₅ receptor antagonist 2-methyl-6-(phenylethynyl)-pyridine on rotarod, locomotor activity and rotational responses in unilateral 6-OHDA-lesioned rats. Eur. J. Pharmacol. 2000, 406, 403-410.

c) Forepaws Tractive Force Measurement:

Dunnet et al.: A laterised grip strength test to evaluate unilateral nigrostriatal lesions in rats. Neurosci. Lett. 1998, 246, 1-4.

The suitability of the compounds according to the invention for the treatment of schizophrenia can be shown in the following animal models:

Catalepsy Test on Rats

The action of test substances on the function of the basal ganglia can be investigated in an animal model using the “catalepsy test on rats” (Sanberg et al., Behav. Neurosci. 1988, 102, 748-759). Catalepsy is remaining in a certain body position, accompanied by increased muscle tone. If a normal animal is brought into an unusual position, it changes its body posture within a few seconds, but a cataleptic animal remains for a relatively long time in the imposed posture. The period of time which elapses up to the correction of an imposed position can be used as a measure of the intensity of catalepsy. In a sufficiently high dose, the antipsychotic haloperidol also induces cataleptic behavior (e.g. Chartoff et al., J. Pharmacol. Exp. Therap. 291, 531-537). In EP-A 1 250 923, it is described that the selective PDE10 inhibitor papaverine induces a potentiation of haloperidol catalepsy.

The action of the selective PDE10 inhibitors is investigated in the animal model mentioned. A low dose of haloperidol (0.3 mg/kg s.c.) is given on its own 30 min before the catalepsy test or administered together with the compound. In order to measure the cataleptic behavior, both forepaws of the rat are put onto a block of wood of 9 cm height and 5.5 cm width×5.5 cm depth. The time which elapses until an animal pulls its forepaws down from the block again is recorded as the duration of catalepsy. All rats are taken from the block of wood after 60 seconds at the latest. The data acquired from each treatment group (10 animals in each case) are analyzed statistically by means of variance analysis (ANOVA).

The intraperitoneal administration of 3 mg/kg of example 16 together with haloperidol causes a significant increase in the duration of catalepsy by 103%. The result of this experiment shows that example 16 can change the basal ganglia function in the same manner as the antipsychotic haloperidol.

The new active compounds can be converted in a known manner into the customary formulations, such as tablets, coated tablets, pills, granules, aerosols, syrups, emulsions, suspensions and solutions, using inert, nontoxic, pharmaceutically suitable vehicles or solvents. Here, the therapeutically active compound should in each case be present in a concentration of approximately 0.5 to 90% by weight of the total mixture, i.e. in amounts which are sufficient in order to achieve the dose range indicated.

The formulations are produced, for example, by extending the active compounds using solvents and/or vehicles, if appropriate using emulsifiers and/or dispersants, where, for example, in the case of the use of water as a diluent, organic solvents can optionally be used as auxiliary solvents.

Administration is carried out in the customary manner, preferably orally, transdermally or parenterally, in particular perlingually or intravenously. It can, however, also be carried out by inhalation via the mouth or nose, for example with the aid of a spray, or topically via the skin.

In general, it has proven advantageous to administer amounts of approximately 0.001 to 10, in the case of oral administration preferably approximately 0.005 to 3, mg/kg of bodyweight, to achieve effective results.

In spite of this, it may optionally be necessary to depart from the amounts mentioned, namely depending on the bodyweight or on the type of administration route, on individual behavior toward the medicament, the manner of its formulation and the time or interval at which administration takes place. Thus, in some cases it can be adequate to manage with less than the aforementioned minimum amount, while in other cases the upper limit mentioned must be exceeded. In the case of the administration of relatively large amounts, it may be advisable to divide these into a number of individual doses over the course of the day.

If not stated otherwise, all quantitative data relate to percentages by weight. Solvent ratios, dilution ratios and concentration data of liquid/liquid solutions in each case relate to the volume. The statement “w/v” means “weight/volume”. For instance, “10% w/v”: 100 ml of solution or suspension contain 10 g of substance.

Abbreviations:

abs. absolute
ACN acetonitrile
aq. aqueous
Bn benzyl
Boc tert-butoxycarbonyl
BSA bovine serum albumin
CDI N,N′-carbonyldiimidazole
CH cyclohexane
DBU 1,8-diazabicyclo[5.4.0]undec-7-ene
TLC thin-layer chromatography
DCI direct chemical ionization (in MS)
DCM dichloromethane
DIC diisopropylcarbodiimide
DIEA N,N-diisopropylethylamine
DMA N,N-dimethylacetamide
DMAP 4-N,N-dimethylaminopyridine
DMF N,N-dimethylformamide
DMSO dimethyl sulfoxide
of th. of theory
EDC N′-(3-dimethylaminopropyl)-N-ethylcarbodiimide×HCl
EDTA ethylenediamine tetra-acetic acid
EA ethyl acetate (acetic acid ethyl ester)
EI electron impact ionization (in MS)
Eq equivalent(s)
ESI electrospray ionization (in MS)
M.p. melting point
sat. saturated
HATU O-(7-azabenzotriazol-1-yl)-N,N,N′,N′-tetramethyluronium hexafluorophosphate
HBTU O-(benzotriazol-1-yl)-N,N,N′,N′-tetramethyluronium hexafluorophosphate
HOBt 1-hydroxyl-1H-benzotriazole×H₂O
HPLC High-pressure, high-performance liquid chromatography
Conc. concentrated
B.p. boiling point
LC-MS liquid chromatography-coupled mass spectroscopy
LDA lithium N,N-diisopropylamide
Lit. literature (reference)
Soln. solution
MW molecular weight
MS mass spectroscopy
NMR nuclear magnetic resonance spectroscopy
PyBOP benzotriazol-1-yloxy-tris(pyrrolidino)phosphonium hexafluorophosphate
RF reflux
R_{f} retention index (in TLC)
RP reverse phase (in HPLC)
RT room temperature
Rₜ retention time (in HPLC)
TBTU O-(benzotriazol-1-yl)-N,N,N′,N′-tetramethyluronium tetrafluoroborate
TEA triethylamine
TFA trifluoroacetic acid
TRIS tris-(hydroxymethyl)aminomethane
THF tetrahydrofuran
v/v volume-to-volume ratio (of a solution)
dil. dilute
aq. aqueous
dec. decomposition
   HPLC and LC-MS Methods:
   Method 1 (LCMS)

Instrument: Micromass Quattro LCZ, HP1100; column: Symmetry C18, 50 mm×2.1 mm, 3.5 μm; eluent A: acetonitrile+0.1% formic acid, eluent B: water+0.1% formic acid; gradient: 0.0 min 10% A→4.0 min 90% A→6.0 min 90% A; oven: 40° C.; flow: 0.5 ml/min; UV detection: 208-400 nm.

Method 2 (LCMS)

Instrument: Finnigan MAT 900S, TSP: P4000,AS3000,UV3000HR; column: Symmetry C 18, 150 mm×2.1 mm, 5.0 μm; eluent C: water, eluent B: water+0.3 g 35% strength HCl, eluent A: acetonitrile; gradient: 0.0 min 2% A→2.5 min 95% A→5 min 95% A; oven: 70° C.; flow: 1.2 ml/min; UV detection: 210 nm.

Method 3 (LCMS)

Apparatus type MS: Micromass ZQ; apparatus type HPLC: Waters Alliance 2790; column: Symmetry C 18, 50 mm×2.1 mm, 3.5 μm; eluent B: acetonitrile+0.05% formic acid, eluent A: water+0.05% formic acid; gradient: 0.0 min 10% B→3.5 min 90% B→5.5 min 90% B; oven: 50° C.; flow: 0.8 ml/min; UV detection: 210 nm.

Method 4 (LCMS)

Instrument: Micromass Quattro LCZ, HP1100; column: Symmetry C18, 50 mm×2.1 mm, 3.5 μm; eluent A: water+0.05% formic acid, eluent B: acetonitrile+0.05% formic acid; gradient: 0.0 min 90% A→4.0 min 10% A→6.0 min 10% A; oven: 40° C.; flow: 0.5 ml/min; UV detection: 208-400 nm.

Method 5 (LCMS)

Instrument: Micromass Platform LCZ, HP1100; column: Symmetry C18, 150 mm×2.1 mm, 5 μm; eluent A: water+0.05% formic acid, eluent B: acetonitrile+0.05% formic acid; gradient: 0.0 min 90% A→ 9.0 min 10% A→10.0 min 10% A; oven: 40° C.; flow: 0.5 ml/min; UV detection: 208-400 nm.

Method 6 (LCMS)

Instrument: Micromass Platform LCZ, HP1100; column: Symmetry C18, 50 mm×2.1 mm, 3.5 μm; eluent A: water+0.05% formic acid, eluent B: acetonitrile+0.05% formic acid; gradient: 0.0 min 90% A→4.0 min 10% A→6.0 min 10% A; oven: 40° C.; flow: 0.5 ml/min; UV detection: 208-400 nm.

Method 7 (LCMS)

Instrument: Waters Alliance 2790 LC; column: Symmetry C18, 50 mm×2.1, 3.51 μm; eluent A: water+0.1% formic acid, eluent B: acetonitrile+0.1% formic acid; gradient: 0.0 min 5% B→5.0 min 10% B→6.0 min 10% B; temperature: 50° C.; flow: 1.0 ml/min; UV detection: 210 nm.

Method 8 (HPLC)

Instrument: HP 1100 with DAD detection; column: Kromasil RP-18, 60 mm×2 mm, 3.5 μm; eluent: A=5 ml HClO₄/l H₂O, B=ACN; gradient: 0 min 2% B, 0.5 min 2% B, 4.5 min 90% B, 6.5 min 90% B; flow: 0.75 ml/min; temp.: 30° C.; detection UV 210 nm.

Starting Compounds

### EXAMPLE 1A

### 3-Thiophenecarboximidamide hydrochloride

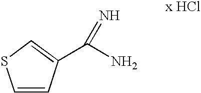

29.40 g (549.7 mmol) of ammonium chloride are suspended in 200 ml of toluene under an argon atmosphere in a three-necked flask having a thermometer, condenser, dropping funnel and mechanical stirrer and cooled at 0° C. using petroleum ether/dry ice. 247 ml (494 mmol) of a 2 molar solution of trimethylaluminum in hexane are added dropwise, and the mixture is stirred at room temperature until evolution of gas is no longer observed (about 1.5 hours). 20.0 g (183 mmol) of 3-thiophene-carbonitrile are subsequently rapidly added to this mixture, and the reaction mixture is stirred overnight at 80° C.

After cooling, the mixture is treated dropwise with methanol at 0° C. and subsequently stirred vigorously at room temperature. The mixture is filtered off with suction and the residue is washed 5 times with 60 ml each of methanol. The filtrate is concentrated, and the residue is suspended using dichloromethane/methanol (10:1).

The insoluble residue of ammonium chloride is filtered off and the filtrate is concentrated again and dried.

Yield: 19.28 g (64% of th.)

LC/MS (method 4): Rₜ=0.48 min

MS (EI): m/z=126 (M+H—HCL)⁺

### EXAMPLE 2A

### Imino(5-methyl-3-pyridinyl)methanaminium chloride

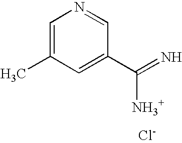

Preparation analogously to example 1A using 13.59 g (254.0 mmol) of ammonium chloride, 127 ml (254 mmol) of a 2 molar solution of trimethylaluminum in hexane and 9.60 g (63.51 mmol) of methyl 5-methylnicotinate.

Yield: 8.07 g (74% of th.)

LC/MS (method 3): Rₜ=0.37 min

MS (EI): m/z=135 (M+H—HCL)⁺

### EXAMPLE 3A

### Imino(6-methyl-3-pyridinyl)methanaminium chloride

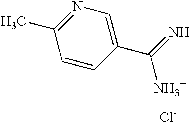

Preparation analogously to example 1A using 14.15 g (264.6 mmol) of ammonium chloride, 132 ml (264 mmol) of a 2 molar solution of trimethylaluminum in hexane and 10.0 g (66.15 mmol) of methyl 6-methylnicotinate.

Yield: 11.20 g (88% of th.)

LC/MS (method 4): Rₜ=2.01 min

MS (EI): In/z=135 (M+H—HCL)⁺

### EXAMPLE 4A

### Ethyl 3-(acetylamino)-2-oxobutanoate

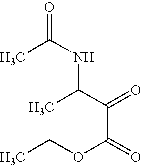

A solution of N-acetyl-alanine (4.92 g, 37.5 mmol), 9.10 ml of pyridine and 150 mg of DMAP in 200 ml THF is brought to boiling. At boiling heat, 8.6 ml (10.5 g, 75 mmol) of ethyl oxalyl chloride are added dropwise, and after addition is complete the mixture is stirred for a further 3 h at boiling heat. After cooling, the reaction mixture is added to 600 ml of ice water, extracted with ethyl acetate (4×150 ml), and the combined organic phases are washed with 200 ml of saturated sodium chloride solution, dried over sodium sulfate and concentrated. The material obtained is dissolved in ethanol without delay and reacted further.

### EXAMPLE 5A

### N-{1-[5-Oxo-3-(2-pyridinyl)-4,5-dihydro-1,2,4-triazin-6-yl]ethyl}acetamide

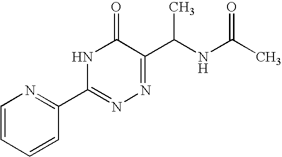

A solution of 9.60 g (60.91 mmol) 2-pyridinecarboximidamide hydrochloride in ethanol is treated with 3.66 g (3.56 ml; 73.10 mmol) of hydrazine hydrate. The mixture is stirred for one hour at room temperature. Subsequently, 17.10 g (91.37 mmol) of ethyl 3-(acetylamino)-2-oxobutanoate (from example 4A, dissolved in ethanol) are added. For better solubility, some dimethyl sulfoxide is added thereto. The reaction mixture is stirred for 4 h at 70-80° C. The mixture is cooled, concentrated and the residue is purified by flash chromatography (eluent: dichloromethane/methanol 30:1-1:1).

Yield: 12.44 g (32% of th.).

LC/MS (method 1): Rₜ=0.37 min

MS (EI): m/z=282 (M+Na)⁺

### EXAMPLE 6A

### N-{1-[3-(2-Furyl)-5-oxo-4,5-dihydro-1,2,4-triazin-6-yl]ethyl}acetamide

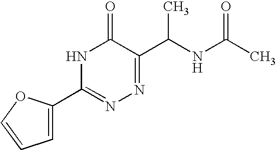

Preparation analogously to example 5A using 10.0 g (68.22 mmol) of 2-furan-carboximidamide hydrochloride, 4.10 g (3.98 ml; 81.87 mmol) of hydrazine hydrate and 19.16 g (102.34 mmol) of ethyl 3-(acetylamino)-2-oxobutanoate from example 4A.

Yield: 5.34 g (28% of th.).

LC/MS (method 1): Rₜ=0.36 min

MS (ESIpos): m/z=249 (M+H)⁺.

### EXAMPLE 7A

### N-{1-[3-(2-Methyl-1,3-thiazol-5-yl)-5-oxo-4,5-dihydro-1,2,4-triazin-6-yl]ethyl}-acetamide

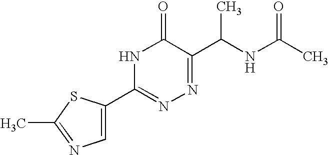

Preparation analogously to example 5A using 10.90 g (61.35 mmol) of 2-methyl-1,3-thiazole-5-carboximidamide hydrochloride, 3.69 g (3.58 ml; 73.62 mmol) of hydrazine hydrate and 17.23 g (92.03 mmol) of ethyl 3-(acetylamino)-2-oxobutanoate from Example 4A.

Yield: 4.69 g (27% of th.).

LC/MS (method 2): Rₜ=1.52 min

MS (EI): m/z=280 (M+H)⁺

¹H-NMR (200 MHz, DMSO-d₆): δ=1.29 (s, 3H), 1.32 (s, 3H), 1.83 (s, 3H), 5.01 (quint, 1H), 5.75 (s, 1H), 8.22 (d, 1H), 8.41 (s, 1H).

### EXAMPLE 8A

### N-{1-[5-Oxo-3-(3-thienyl)-4,5-dihydro-1,2,4-triazin-6-yl]ethylacetamide

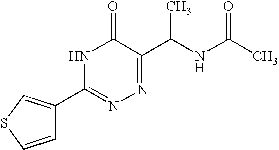

Preparation analogously to example 5A using 19.23 g (118.23 mmol) of 3-thiophenecarboximidamide hydrochloride from example 1A, 7.10 g (6.90 ml; 141.88 mmol) of hydrazine hydrate and 39.84 g (212.82 mmol) of ethyl 3-(acetylamino)-2-oxobutanoate from example 4A.

Yield: 4.60 g (15% of th.).

LC/MS (method 1): Rₜ=1.17 min

MS (EI): m/z=287 (M+Na)+

¹H-NMR (400 MHz, MeOH-d₄): δ=1.46 (d, 3H), 1.98 (s, 3H), 5.17 (q, 1H), 7.63 (dd, 1H), 7.71 (dd, 1H), 8.38 (dd, 1H).

Preparation Analogously to Example 5A:

| Example | Structure | Analytical data |
|---|---|---|
| 9A | 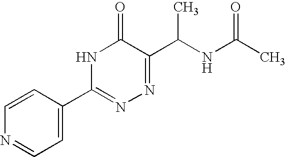 | LC/MS (method 1): Rₜ = 0.38 min MS (EI): m/z = 282 (M + Na)⁺¹H-NMR (300 MHz, DMSO-d₆): δ = 1.34(d, 3H), 1.85(s, 3H), 5.06(quint, 1H), 7.98(dd, 2H), 8.79(dd, 2H). |
| 10A | 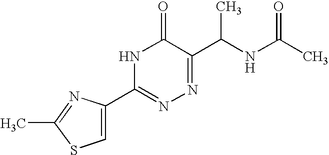 | LC/MS (method 1): Rₜ = 1.22 min MS (EI): m/z = 302 (M + Na)⁺¹H-NMR (300 MHz, CDCl₃): δ = 1.52(d, 3H), 2.00(s, 3H), 3.18(s, 3H), 5.18-5.32(m, 1H), 6.82(d, 1H), 8.35(s, 1H), 11.30(br. s, 1H). |
| 11A | 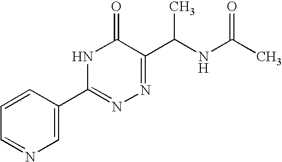 | LC/MS (method 1): Rₜ = 0.48 min MS (EI): m/z = 260 (M + H)⁺¹H-NMR (300 MHz, MeOH-d₄): δ = 1.48(d, 3H), 1.97(s, 3H), 5.19(q, 1H), 7.63(dd, 1H), 8.42(dt, 1H), 8.79(dd, 1H), 9.15(d, 1H). |
| 12A | 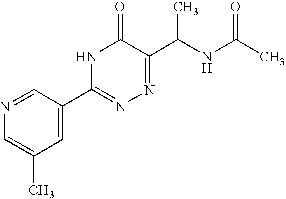 | LC/MS (method 5): Rₜ = 0.41 min MS (EI): m/z = 274 (M + H)⁺¹H-NMR (300 MHz, DMSO-d₆): δ = 1.33(d, 3H), 1.85(s, 3H), 2.39(s, 3H), 5.06(quint, 1H), 8.22(s, 1H), 8.61(d, 1H), 8.99(d, 1H). |
| 13A | 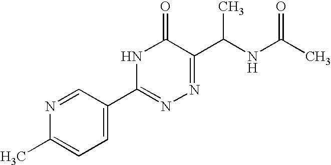 | LC/MS (method 5): Rₜ = 0.33 min MS (EI): m/z = 272 (M − H)⁺¹H-NMR (200 MHz, DMSO-d₆): δ = 1.34(d, 3H), 1.84(s, 3H), 2.57(s, 3H), 5.03(quint, 1H), 7.47(d, 1H), 8.27(d, 2H), 9.05(d, 1H), 13.70(br. s, 1H). |

### EXAMPLE 14A

### 6-(1-Aminoethyl)-3-(3-pyridinyl)-1,2,4-triazin-5 (4H)-one

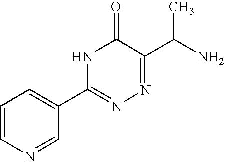

A solution of 2.43 g (9.37 mmol) of N-{1-[5-oxo-3-(3-pyridinyl)-4,5-dihydro-1,2,4-triazin-6-yl]ethyl}acetamide from example 11A in 50 ml of 2 molar hydrochloric acid is heated for 3 hours at 100° C. Subsequently, the solution is concentrated under reduced pressure, and the residue is taken up in methanol and rendered alkaline with 1 molar sodium hydroxide solution. The solvent is removed under reduced pressure and the residue is purified by flash chromatography (eluent: dichloromethane/methanol 20:2-10:1-5:1).

Yield: 1.25 g (55% of th.).

LC/MS (method 5): Rₜ=0.35 min

MS (EI): m/z=217 (M-H)⁺

¹H-NMR (200 MHz, DMSO-d₆): δ=1.48 (d, 3H), 4.44 (q, 1H), 7.39-7.79 (m, 3H), 8.49 (dt, 1H), 8.63 (dd, 1H), 9.34 (s, 1H).

### EXAMPLE 15A

### 5,7-Dimethyl-2-(2-pyridinyl)imidazo[5,1-f][1,2,4]triazin-4(3H)-one

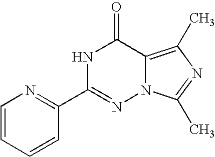

A solution of 1.70 g (6.56 mmol) of N-{1-[5-oxo-3-(2-pyridinyl)-4,5-dihydro-1,2,4-triazin-6-yl]ethyl}acetamide from example 5A in 20 ml of 1,2-dichloroethane is treated with 3.02 g (1.83 ml; 19.67 mmol) of phosphoryl chloride. The mixture is heated under reflux for 3 hours and cooled again. 5 ml of aqueous sodium hydrogencarbonate solution are added thereto. The solvent is removed under reduced pressure and toluene is added to remove the remaining water and the mixture is again concentrated to dryness. The residue is purified by flash chromatography (dichloromethane/methanol 10:1) and the clean fraction is stirred with diethyl ether/toluene 10:1. The resulting crystals are filtered off with suction and dried.

Yield: 175 mg (10% of th.).

LC/MS (method 1): Rₜ=2.40 min

MS (EI): m/z=242 (M+H)⁺

¹H-NMR (200 MHz, DMSO-d₆): δ=2.47 (s, 3H), 2.56 (s, 3H), 7.65 (t, 1H), 8.05 (t, 1H), 8.26 (d, 1H), 8.74 (d, 1H), 11.22 (br. s, 1H).

### EXAMPLE 16A

### 2-(2-Furyl)-5,7-dimethylimidazo[5,1-f][1,2,4]triazin-4(3H)-one

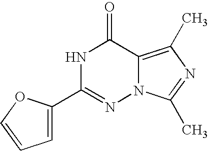

Preparation analogously to example 15A using 2.00 g (8.06 mmol) of N-{1-[3-(2-furyl)-5-oxo-4,5-dihydro-1,2,4-triazin-6-yl]ethyl}acetamide from example 6A, 30 ml of 1,2-dichloromethane and 3.71 g (2.25 ml; 24.17 mmol) of phosphoryl chloride.

Yield: 1.07 g (58% of th.).

LC/MS (method 2): Rₜ=1.51 min

MS (EI): m/z=231 (M+H)⁺

¹H-NMR (300 MHz, DMSO-d₆): δ=2.46 (s, 3H), 2.50 (s, 3H), 6.73 (dd, 1H), 7.56 (d, 1H), 7.98 (d, 1H), 11.85 (br. s, 1H).

### EXAMPLE 17A

### 5,7-Dimethyl-2-(2-methyl-1,3-thiazol-5-yl)imidazo[5,1-f][1,2,4]triazin-4(3H)-one

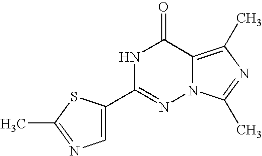

Preparation analogously to example 15A using 2.00 g (7.16 mmol) of N-{1-[3-(2-methyl-1,3-thiazol-5-yl)-5-oxo-4,5-dihydro-1,2,4-triazin-6-yl]ethyl}acetamide from example 7A, 1,2-dichloromethane and 3.29 g (2.00 ml; 21.48 mmol) of phosphoryl chloride.

Yield: 362 mg (19% of th.).

LC/MS (method 2): Rₜ=1.60 min

MS (EI): m/z=262 (M+H)⁺

¹H-NMR (300 MHz, DMSO-d₆): δ=2.45 (s, 3H), 2.46 (s, 3H), 2.70 (s, 3H), 8.52 (s, 1H), 12.01 (br. s, 1H).

Preparation Analogously to Example 15A:

| Example | Structure | Analytical data |
|---|---|---|
| 18A | 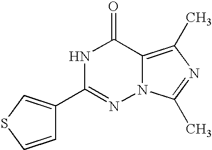 | LC/MS (method 1): Rₜ = 2.23 min MS (EI): m/z = 247 (M + H)⁺¹H-NMR (300 MHz, MeOH-d₄): δ = 2.71(s, 3H), 2.82(s, 3H), 7.64(dd, 1H), 7.73(dd, 1H), 8.37(dd, 1H). |
| 19A | 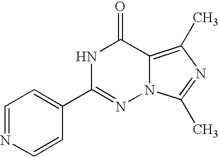 | LC/MS (method 2): Rₜ = 1.81 min MS (EI): m/z = 242 (M + H)⁺ |
| 20A | 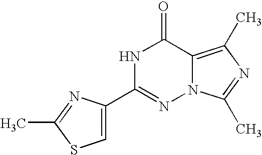 | LC/MS (method 1): Rₜ = 2.16 min MS (EI): m/z = 262 (M + H)⁺¹H-NMR (200 MHz, DMSO-d₆): δ = 2.53(s, 3H), 2.61(s, 3H), 2.76(s, 3H), 8.47(s, 1H), 11.94(br. s, 1H). |
| 21A | 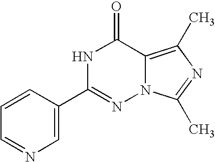 | LC/MS (method 1): Rₜ = 0.86 min MS (EI): m/z = 242 (M + H)⁺¹H-NMR (500 MHz, DMSO-d₆): δ = 2.47(s, 3H), 2.52(s, 3H), 7.58(dd, 1H), 8.33(dt, 1H), 8.76(d, 1H), 9.13(s, 1H), 12.02(br. s, 1H). |
| 22A | 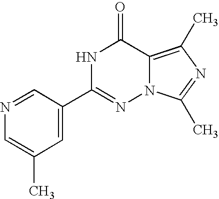 | LC/MS (method 3): Rₜ = 0.36 min MS (EI): m/z = 256 (M + H)⁺¹H-NMR (300 MHz, DMSO-d₆): δ = 2.39(s, 3H), 2.47(s, 3H), 2.52(s, 3H), 8.17(s, 1H), 8.60(s, 1H), 8.93(s, 1H), 11.91(br. s, 1H). |
| 23A | 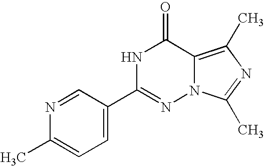 | LC/MS (method 5): Rₜ = 2.26 min MS (EI): m/z = 256 (M + H)⁺HPLC (method 8): Rₜ = 4.30 min. ¹H-NMR (300 MHz, DMSO-d₆): δ = 2.47(s, 3H), 2.51(s, 3H), 3.01(s, 3H), 7.42(d, 1H), 8.22(dd, 1H), 9.00(d, 1H), 11.98(br. s, 1H). |

### EXAMPLE 24A

### 7-Isopropyl-5-methyl-2-(3-pyridinyl)imidazo[5,1-f][1,2,4]triazin-4(3H)-one

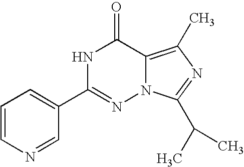

758 mg (7.50 mmol) of triethylamine are added to a solution of 543 mg (2.50 mmol) of 6-(1-aminoethyl)-3-(3-pyridinyl)-1,2,4-triazin-5(4H)-one from example 14A in 12 ml of dimethylformamide. The mixture is cooled to 0° C. 532.76 mg (5.00 mmol) of isobutyryl chloride are added dropwise thereto and the mixture is stirred for 3 hours at RT. Subsequently, the solvent is removed in vacuo and the residue is dried in a high vacuum. The residue is dissolved in 12 ml of dioxane and treated with 1150 mg (7.50 mmol) of phosphoryl chloride; the reaction mixture is heated for 2 hours to 80° C. On cooling, sodium hydrogencarbonate solution is added dropwise until evolution of gas no longer occurs. The mixture is then rendered alkaline using 1 molar sodium hydroxide solution (about pH 10) and extracted with dichloromethane. The organic phase is dried over magnesium sulfate, filtered and the solvent is removed under reduced pressure. The residue is purified by flash chromatography (dichloromethane/methanol 20:1).

Yield: 347 mg (52% of th.).

LC/MS (method 1): Rₜ=2.90 min

MS (EI): m/z=270 (M+H)⁺

¹H-NMR (300 MHz, DMSO-d₆): δ=1.32 (d, 6H), 2.45 (s, 3H), 3.49 (sept., 1H), 7.58 (dd, 1H), 8.32 (dt, 1H), 8.75 (dd, 1H), 9.12 (d, 1H), 11.98 (br. s, 1H).

### EXAMPLE 25A

### 5,7-Dimethyl-2-(2-pyridinyl)-4-(1H-1,2,4-triazol-1-yl)imidazo[5,1-f][1,2,4]triazine

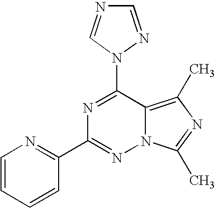

228 mg (0.14 ml; 1.49 mmol) of phosphoryl chloride are added dropwise to a solution of 120 mg (0.50 mmol) of 5,7-dimethyl-2-(2-pyridinyl)imidazo[5,1-f]-[1,2,4]triazin-4(3H)-one from example 15A in 3 ml of dry pyridine at RT, and the mixture is stirred for 90 minutes. Subsequently, 309.2 mg (4.48 mmol) of 1,2,4-triazole is added, and the mixture is stirred at RT overnight after addition is complete. The mixture is cautiously treated with 1 ml of water, and stirred for 30 minutes. The reaction mixture is concentrated, the residue is treated with 20 ml of aqueous sodium hydrogencarbonate solution and the mixture is extracted with dichloromethane. The organic phase is dried and the solvent is removed in vacuo. The residue is purified by flash chromatography (eluent: dichloromethane/methanol 10:1). The clean fraction is stirred with diethyl ether, and the crystals are filtered off with suction and dried.

Yield: 68 mg (47% of th.)

LC/MS (method 1): Rₜ=2.80 min

MS (EI): m/z=293 (M+H)⁺

¹H-NMR (300 MHz, CDCl₃): δ=2.89 (s, 3H), 2.91 (s, 3H), 7.44-7.52 (m., 1H), 7.87-7.95 (m, 1H), 8.27 (s, 1H), 8.40 (d, 1H), 8.87 (d, 1H), 9.42 (s, 1H).

### EXAMPLE 26A

### 2-(2-Furyl)-5,7-dimethyl-4-(1H-1,2,4-triazol-1-yl)imidazo[5,1-f][1,2,4]triazine

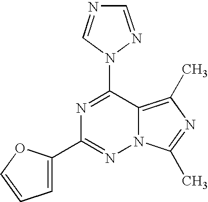

Preparation analogously to example 25A using 810 mg (3.52 mmol) of 2-(2-furyl)-5,7-dimethylimidazo[5,1-f][1,2,4]triazin-4(3H)-one from example 16A, 10 ml of pyridine, 1618 mg (10.55 mmol) of phosphoryl chloride and 2187 mg (31.66 mmol) of 1,2,4-triazole.

Yield: 230 mg (23% of th.)

LC/MS (method 1): Rₜ=3.30 min

MS (EI): m/z=282 (M+H)⁺

¹H-NMR (300 MHz, CDCl₃): δ=2.80 (s, 3H), 2.86 (s, 3H), 6.61 (dd., 1H), 7.32 (dd, 1H), 7.65-7.69 (m, 1H), 8.25 (s, 1H), 9.31 (s, 1H).

Preparation Analogously to Example 25A:

| Example | Structure | Analytical data |
|---|---|---|
| 27A | 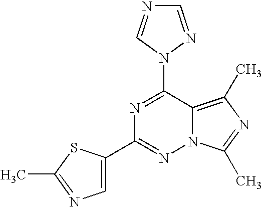 | LC/MS (method 1): Rₜ = 3.30 min MS (EI): m/z = 313 (M + H)⁺¹H-NMR (400 MHz, CDCl₃): δ = 2.76(s, 3H), 2.80(s, 3H), 2.87(s, 3H), 8.26(s, 1H), 8.42(s, 1H), 9.32(s, 1H). |
| 28A | 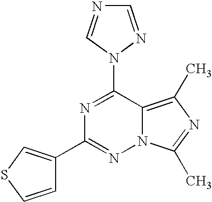 | LC/MS (method 1): Rₜ = 3.78 min MS (EI): m/z = 298 (M + H)⁺¹H-NMR (200 MHz, CDCl₃): δ = 2.79(s, 3H), 2.87(s, 3H), 7.43(dd, 1H), 7.84(dd, 1H), 8.23-8.30(m, 2H), 9.34(s, 1H). |
| 29A | 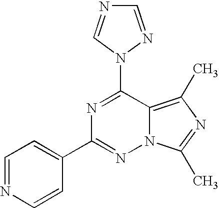 | LC/MS (method 1): Rₜ = 2.66 min MS (EI): m/z = 293 (M + H)⁺¹H-NMR (300 MHz, CDCl₃): δ = 2.84(s, 3H), 2.92(s, 3H), 8.19-8.25(m, 2H), 8.29(s, 1H), 8.82(dd, 2H), 9.40(s, 1H). |
| 30A | 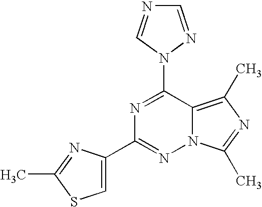 | LC/MS (method 6): Rₜ = 3.03 min MS (EI): m/z = 313 (M + H)⁺HPLC (method 8): Rₜ = 3.38 min. ¹H-NMR (200 MHz, DMSO-d₆): δ = 2.69(s, 3H), 2.75(s, 3H), 2.78(s, 3H), 8.56(s, 1H), 9.45(s, 1H), 9.88(s, 1H). |
| 31A | 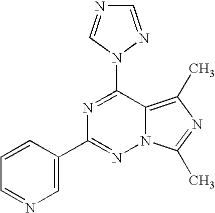 | LC/MS (method 6): Rₜ = 2.83 min MS (EI): m/z = 293 (M + H)⁺¹H-NMR (200 MHz, CDCl₃): δ = 2.83(s, 3H), 2.91(s, 3H), 7.47(dd, 1H), 8.29(s, 1H), 8.62(dt, 1H), 8.78(dd, 1H), 9.40(s, 1H), 9.58(d, 1H). |
| 32A | 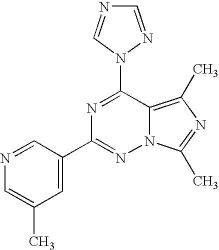 | LC/MS (method 5): Rₜ = 3.50 min MS (EI): m/z = 307 (M + H)⁺¹H-NMR (400 MHz, MeOH-d₄): δ = 2.51(s, 3H), 2.82(s, 3H), 2.88(s, 3H), 8.36(s, 1H), 8.57(d, 2H), 9.34(s, 1H), 9.61(s, 1H). |
| 33A | 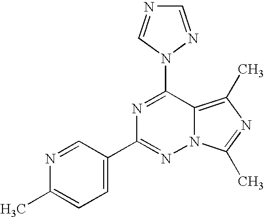 | LC/MS (method 7): Rₜ = 1.87 min MS (EI): m/z = 307 (M + H)⁺¹H-NMR (300 MHz, CDCl₃): δ = 2.67(s, 3H), 2.82(s, 3H), 2.89(s, 3H), 7.31(d, 1H), 8.27(s, 1H), 8.49(dd, 1H), 9.37(s, 1H), 9.45(d, 1H). |
| 34A | 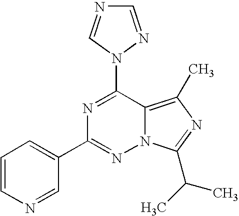 | LC/MS (method 1): Rₜ = 3.71 min MS (EI): m/z = 321 (M + H)⁺¹H-NMR (400 MHz, CDCl₃): δ = 1.52(d, 6H), 2.91(s, 3H), 3.80(sept., 1H), 7.47(dd, 1H), 8.28(s, 1H), 8.61(dt, 1H), 8.77(d, 1H), 9.37(s, 1H), 9.58(d, 1H). |

### EXAMPLE 35A

### 6-Chloro-3-pyridinecarboximidamide hydrochloride

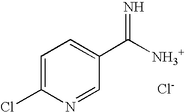

Preparation analogously to example 1A from 14.8 g (86.3 mmol) of methyl 6-chloro-3-pyridinecarboxylate, 11.5 g (215.6 mmol) of ammonium chloride and 108 ml (215.6 mmol) of a 2 molar solution of trimethylaluminum in hexane.

Yield: 9.0 g (67% of th.)

LC/MS (method 6): Rₜ=3.23 min

MS (ESI): m/z=156 (M+H—HCL)⁺

### EXAMPLE 36A

### N-{1-[3-(6-Chloro-3-pyridinyl)-5-oxo-4,5-dihydro-1,2,4-triazin-6-yl]ethyl}acetamide

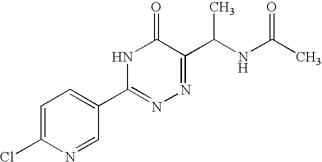

Preparation analogously to example 5A from 9.0 g (46.9 mmol) of 6-chloro-3-pyridinecarboximidamide hydrochloride from example 35A, 2.74 ml (2.82 g; 56.2 mmol) of hydrazine hydrate and 13.2 g (70.3 mmol) of ethyl 3-(acetylamino)-2-oxobutanoate from example 4A.

Yield: 2.20 g (16% of th.).

LC/MS (method 4): Rₜ=2.24 min

MS (ESI): m/z=294 (M+H)⁺.

¹H-NMR (300 MHz, DMSO-d₆): δ=1.35 (d, 3H), 1.84 (s, 3H), 5.05 (quint., 1H), 7.77 (d, 1H), 8.23 (d, 1H), 8.42 (dd, 1H), 9.01 (d, 1H).

### EXAMPLE 37A

### 2-(6-Chloro-3-pyridinyl)-5,7-dimethylimidazo[5,1-f][1,2,4]triazin-4(3H)-one

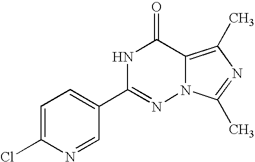

Preparation analogously to example 15A using 2.20 g (7.49 mmol) of N-(1-[3-(6-chloro-3-pyridinyl)-5-oxo-4,5-dihydro-1,2,4-triazin-6-yl]ethyl}acetamide from example 36A and 2.1 ml (22.5 mmol) of phosphoryl chloride in 50 ml of dioxane.

Yield: 719 mg (35% of th.).

LC/MS (method 3): Rₜ=1.75 min

MS (ESI): m/z=276 (M+H)⁺

¹H-NMR (200 MHz, DMSO-d₆): δ=2.47 (s, 3H), 2.52 (s, 3H), 7.73 (d, 1H), 8.39 (dd, 1H), 8.97 (d, 1H), 12.1 (br.s, 1H).

### EXAMPLE 38A

### 2-(6-Chloro-3-pyridinyl)-5,7-dimethyl-4-(1H-1,2,4-triazol-1-yl)imidazo[5,1-f]-[1,2,4]triazine

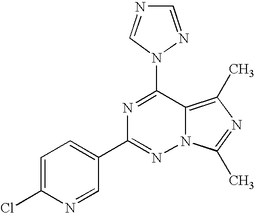

Preparation analogously to example 25A from 100 mg (0.36 mmol) of 2-(6-chloro-3-pyridinyl)-5,7-dimethylimidazo[5,1-f][1,2,4]triazin-4(3H)-one from example 37A, 0.10 ml (1.09 mmol) of phosphoryl chloride, 301 mg (4.35 mmol) of 1,2,4-triazole and 0.59 ml (7.2 mmol) of pyridine in 5 ml of dioxane.

Yield: 73 mg (62% of th.)

LC/MS (method 3): Rₜ=2.63 min

MS (ESI): m/z=327 (M+H)⁺

¹H-NMR (300 MHz, CDCl₃): δ=2.84 (s, 3H), 2.92 (s, 3H), 7.49 (d, 1H), 8.29 (m, 1H), 8.58 (dd, 1H), 9.35 (d, 1H), 9.37 (m, 1H).

### EXAMPLE 39A

### 2-(6-Methoxy-3-pyridinyl)-5,7-dimethylimidazo[5,1-f][1,2,4]triazin-4(3H)-one

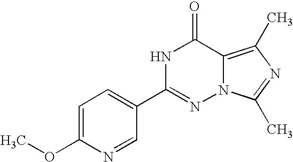

3 ml of anhydrous methanol are introduced under an argon atmosphere and treated with 56 mg (2.45 mmol) of sodium. After evolution of gas is complete, 134 mg (0.49 mmol) of 2-(6-chloro-3-pyridinyl)-5,7-dimethylimidazo[5,1-f][1,2,4]triazin-4(3H)-one from example 37A are added, and the reaction mixture is heated overnight at 70° C. After cooling, it is treated with 20 ml of ammonium chloride solution and extracted three times with 20 ml each of dichloromethane. The organic phase is dried over magnesium sulfate and the solvent is subsequently removed in vacuo.

Yield: 56 mg (42% of th.)

LC/MS (method 3): Rₜ=1.55 min

MS (ESI): m/z=272 (M+H)⁺

¹H-NMR (200 MHz, DMSO-d₆): δ=2.47 (s, 3H), 2.51 (s, 3H), 3.93 (s, 3H), 6.98 (d, 1H), 8.26 (dd, 1H), 8.79 (d, 1H), 11.8 (br.s, 1H).

### EXAMPLE 40A

### 2-(6-Methoxy-3-pyridinyl)-5,7-dimethyl-4-(1H-1,2,4-triazol-1-yl)imidazo[5,1-f]-[1,2,4]triazine

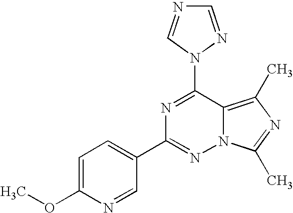

Preparation analogously to example 25A from 215 mg (0.79 mmol) of 2-(6-methoxy-3-pyridinyl)-5,7-dimethylimidazo[5,1-f][1,2,4]triazin-4(3H)-one from example 39A, 0.22 ml (2.38 mmol) of phosphoryl chloride, 657 mg (9.51 mmol) of 1,2,4-triazole and 1.3 ml (15.9 mmol) of pyridine in 10 ml of dioxane.

Yield: 118 mg (46% of th.)

LC/MS (method 3): Rₜ=2.65 min

MS (ESI): m/z=323 (M+H)⁺

¹H-NMR (200 MHz, CDCl₃): δ=2.82 (s, 3H), 2.90 (s, 3H), 4.04 (s, 3H), 6.89 (d, 1H), 8.28 (s, 1H), 8.50 (dd, 1H), 9.18 (d, 1H), 9.36 (s, 1H).

### PREPARATION EXAMPLES

### Example 1

### 5,7-Dimethyl-2-(2-pyridinyl)-4-(3,4,5-trimethoxyphenoxy)imidazo[5,1-f][1,2,4]-triazine

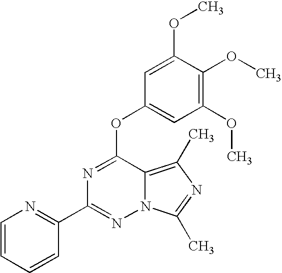

A solution of 52.23 mg (0.28 mmol) of 3,4,5-trimethoxyphenol in 1 ml of tetrahydrofuran is treated with 31.82 mg (0.28 mmol) of potassium tert-butoxide. The mixture is stirred for 10 minutes and 41.45 mg (0.14 mmol) of 5,7-dimethyl-2-(2-pyridinyl)-4-(1H-1,2,4-triazol-1-yl)imidazo[5,1-f][1,2,4]triazine from example 25A are added. The mixture is heated for 5 hours at 65° C. After cooling, the mixture is diluted with 10 ml of dichloromethane and treated with 15 ml of aqueous sodium hydrogencarbonate solution. It is extracted with dichloromethane, the organic phase is dried over sodium sulfate, filtered and the solvent is removed under reduced pressure. The residue is purified by means of preparative HPLC.

Yield: 26 mg (45% of th.)

LC/MS (method 1): Rₜ=2.80 min

MS (EI): m/z=408 (M+H)+

¹H-NMR (300 MHz, CDCl₃): δ=2.75 (s, 3H), 2.84 (s, 3H), 3.88 (s, 6H), 3.91 (s, 3H), 6.65 (s, 2H), 7.32-7.41 (m, 1H), 7.71-7.79 (m, 1H), 8.06 (d, 1H), 8.78 (m, 1H).

### Example 2

### 2-(2-Furyl)-5,7-dimethyl-4-(3,4,5-trimethoxyphenoxy)imidazo[5,1-f][1,2,4]triazine

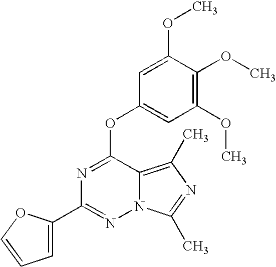

Preparation analogously to example 1 using 123.1 mg (0.67 mmol) of 3,4,5-trimethoxy-phenol, 75 mg (0.67 mmol) of potassium tert-butoxide and 94 mg (0.33 mmol) of 2-(2-furyl)-5,7-dimethyl-4-(1H-1,2,4-triazol-1-yl)imidazo[5,1-f][1,2,4]triazine from example 26A. For workup, the crystals are precipitated using acetonitrile and water, filtered off and dried.

Yield: 111 mg (84% of th.)

LC/MS (method 1): Rₜ=3.80 min

MS (EI): m/z=397 (M+H)+

¹H-NMR (300 MHz, CDCl₃): δ=2.71 (s, 3H), 2.75 (s, 3H), 3.87 (s, 6H), 3.90 (s, 3H), 6.47 (dd, 1H), 6.59 (s, 2H), 6.95 (d, 1H), 7.57 (d, 1H).

Preparation Analogously to Example 1:

| Example | Structure | Analytical data |
|---|---|---|
| 3 | 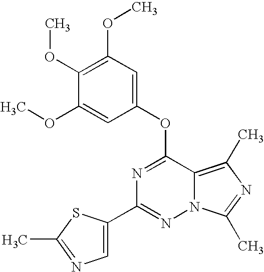 | LC/MS (method 1): Rₜ = 3.83 min MS (EI): m/z = 428 (M + H)⁺¹H-NMR (200 MHz, CDCl₃): δ = 2.66-2.75(m, 9H), 3.88(s, 6H), 3.91(s, 3H), 6.59(s, 2H), 8.12(s, 1H). |
| 4 | 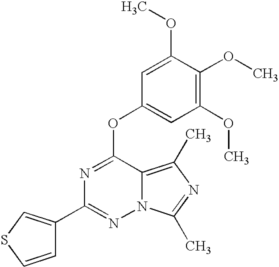 | LC/MS (method 1): Rₜ = 4.31 min MS (EI): m/z = 413 (M + H)⁺¹H-NMR (300 MHz, CDCl₃): δ = 2.72(s, 3H), 2.73(s, 3H), 3.87(s, 6H), 3.91(s, 3H), 6.59(s, 2H), 7.32(dd, 1H), 7.70(dd, 1H), 7.93(dd, 1H). |
| 5 | 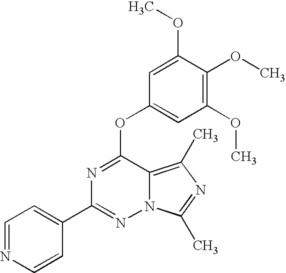 | LC/MS (method 6): Rₜ = 3.39 min MS (EI): m/z = 408 (M + H)⁺¹H-NMR (300 MHz, CDCl₃): δ = 2.74(s, 3H), 2.78(s, 3H), 3.88(s, 6H), 3.92(s, 3H), 6.59(s, 2H), 7.99(d, 2H), 8.69(br. s, 2H). |
| 6 | 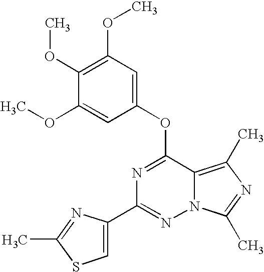 | LC/MS (method 1): Rₜ = 3.58 min MS (EI): m/z = 428 (M + H)⁺¹H-NMR (200 MHz, DMSO-d₆): δ = 2.61(s, 3H), 2.64(s, 3H), 2.72(s, 3H), 3.71(s, 3H), 3.79(s, 6H), 6.84(s, 2H), 7.81(s, 1H). |
| 7 | 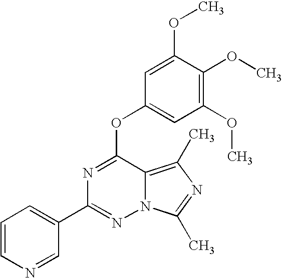 | LC/MS (method 6): Rₜ = 3.57 min MS (EI): m/z = 408 (M + H)⁺¹H-NMR (200 MHz, DMSO-d₆): δ = 2.64(s, 3H), 2.68(s, 3H), 3.72(s, 3H), 3.79(s, 6H), 6.87(s, 2H), 7.54(dd, 1H), 8.34(dt, 1H), 8.69(dd, 1H), 9.17(d, 1H). |
| 8 | 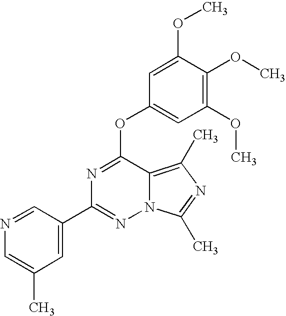 | LC/MS (method 6): Rₜ = 4.20 min MS (EI): m/z = 422 (M + H)⁺¹H-NMR (200 MHz, CDCl₃): δ = 2.39(s, 3H), 2.74(s, 3H), 2.78(s, 3H), 3.88(s, 6H), 3.91(s, 3H), 6.61(s, 2H), 82.3(m, 1H), 8.50(d, 1H), 9.14(d, 1H). |
| 9 | 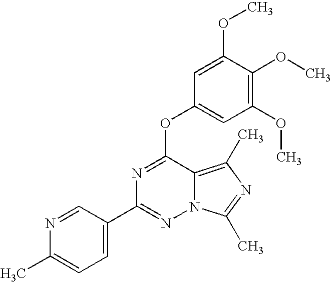 | LC/MS (method 6): Rₜ = 3.80 min MS (EI): m/z = 422 (M + H)⁺¹H-NMR (300 MHz, CDCl₃): δ = 2.68(s, 3H), 2.74(s, 3H), 2.77(s, 3H), 3.87(s, 6H), 3.91(s, 3H), 6.56(s, 2H), 7.24-7.31(m, 1H), 8.38(d, 1H), 9.24(d, 1H). |
| 10 | 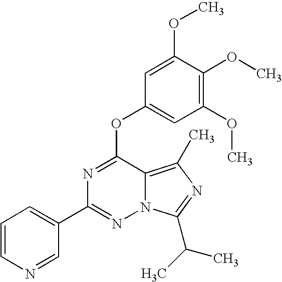 | LC/MS (method 6): Rₜ = 4.70 min MS (EI): m/z = 436 (M + H)⁺¹H-NMR (300 MHz, CDCl₃): δ = 1.49(d, 6H), 2.75(s, 3H), 3.72(quint., 1H), 3.87(s, 6H), 3.91(s, 3H), 6.58(s, 2H), 7.34(dd, 1H), 8.38(dt, 1H), 8.66(d, 1H), 9.36(s, 1H). |

### Example 11

### 2-(2-Furyl)-5,7-dimethyl-N-(3,4,5-trimethoxyphenyl)imidazo[5,1-f][1,2,4]triazin-4-amine

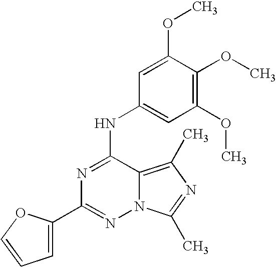

A solution of 128.55 mg (0.70 mmol) of 3,4,5-trimethoxyaniline in 1 ml of tetrahydrofuran is treated with 96.74 mg (0.70 mmol) of potassium carbonate. The mixture is stirred for 10 minutes and 98.68 mg (0.35 mmol) of 2-(2-furyl)-5,7-dimethyl-4-(1H-1,2,4-triazol-1-yl)imidazo[5,1-f][1,2,4]triazine from example 26A are added. The mixture is heated for 48 hours at 90° C. It is treated with toluene and heated under reflux for a further 24 hours. After cooling, the mixture is diluted with 10 ml of dichloromethane and treated with 15 ml of aqueous sodium hydrogencarbonate solution. It is extracted with dichloromethane, the organic phase is dried over sodium sulfate, filtered and the solvent is removed under reduced pressure. The residue is purified by means of preparative HPLC.

Yield: 111 mg (80% of th.)

LC/MS (method 1): Rₜ=3.30 min

MS (EI): m/z=396 (M+H)⁺

¹H-NMR (300 MHz, CDCl₃): δ=2.71 (s, 3H), 2.77 (s, 3H), 3.87 (s, 3H), 3.95 (s, 6H), 6.53 (dd, 1H), 7.04 (br. s, 1H), 7.13 (s, 2H), 7.16 (dd, 1H), 7.56-7.59 (m, 1H).

### Example 12

### 5,7-Dimethyl-2-(2-methyl-1,3-thiazol-5-yl)-N-(3,4,5-trimethoxyphenyl)imidazo-[5,1-f][1,2,4]triazin-4-amine

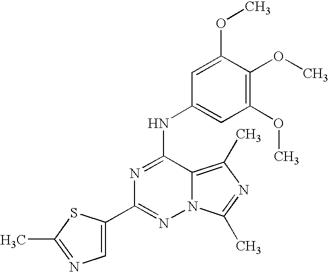

Preparation analogously to example 11 using 70.38 mg (0.19 mmol) of 3,4,5-trimethoxyaniline, 53.1 mg (0.38 mmol) of potassium carbonate and 60 mg (0.19 mmol) of 5,7-dimethyl-2-(2-methyl-1,3-thiazol-5-yl)-4-(1H-1,2,4-triazol-1-yl)-imidazo[5,1-f][1,2,4]triazine from example 27A in 2 ml of DMF at 80° C. For workup, the product is stirred with methanol, filtered, washed with diethyl ether and the crystals are dried.

Yield: 53 mg (65% of th.)

LC/MS (method 1): Rₜ=3.26 min

MS (EI): m/z=427 (M+H)⁺

¹H-NMR (300 MHz, CDCl₃): δ=2.66 (s, 3H), 2.75 (s, 3H), 2.77 (s, 3H), 3.88 (s, 3H), 3.95 (s, 6H), 7.06 (m, 3H), 8.32 (s, 1H).

Preparation Analogously to Example 12:

| Example | Structure | Analytical data |
|---|---|---|
| 13 | 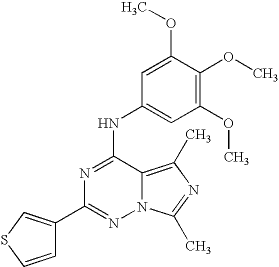 | LC/MS (method 1): Rₜ = 3.70 min MS (EI): m/z = 412 (M + H)⁺¹H-NMR (300 MHz, CDCl₃): δ = 2.68(s, 3H), 2.77(s, 3H), 3.88(s, 3H), 3.93(s, 3H), 7.08(s, 2H), 7.36(dd, 1H), 7.80(dd, 1H), 8.12(dd, 1H), 8.20(br. s, 1H). |
| 14 | 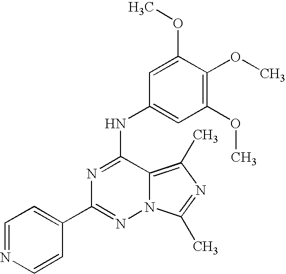 | LC/MS (method 1): Rₜ = 2.85 min MS (EI): m/z = 407 (M + H)⁺¹H-NMR (300 MHz, CDCl₃): δ = 2.72(s, 3H), 2.79(s, 3H), 3.89(s, 3H), 3.94(s, 6H), 7.08(s, 2H), 7.12(br.s, 1H), 8.18(m, 2H), 8.73(m, 1H). |
| 15 | 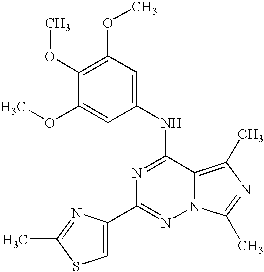 | LC/MS (method 1): Rₜ = 3.18 min MS (EI): m/z = 427 (M + H)⁺¹H-NMR (200 MHz, DMSO-d₆): δ = 2.57(s, 3H), 2.70(s, 3H), 2.72(s, 3H), 3.68(s, 3H), 3.83(s, 6H), 7.37(s, 2H), 8.05(s, 1H), 8.71(br. s, 1H). |
| 16 | 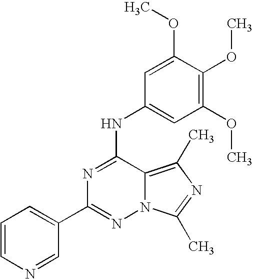 | LC/MS (method 7): Rₜ = 2.51 min MS (EI): m/z = 407 (M + H)⁺¹H-NMR (300 MHz, CDCl₃): δ = 2.71(s, 3H), 2.79(s, 3H), 3.88(s, 3H), 3.94(s, 6H), 7.10(s, 2H), 7.26(s, 1H), 7.37(dd, 1H), 8.57(dt, 1H), 8.69(dd, 1H), 9.54(br. s, 1H). |
| 17 | 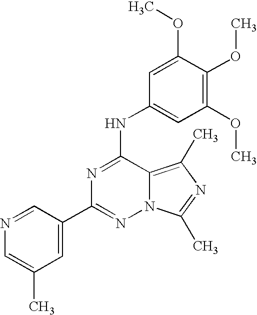 | LC/MS (method 6): Rₜ = 3.70 min MS (EI): m/z = 421 (M + H)⁺¹H-NMR (200 MHz, CDCl₃): δ = 2.42(s, 3H), 2.73(s, 3H), 2.80(s, 3H), 3.88(s, 3H), 3.96(s, 6H), 7.11(m, 3H), 8.39(m, 1H), 8.52(m, 1H), 9.35(m, 1H). |
| 18 | 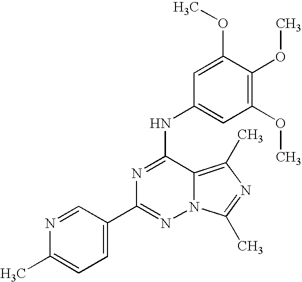 | LC/MS (method 7): Rₜ = 2.20 min MS (EI): m/z = 421 (M + H)⁺¹H-NMR (300 MHz, CDCl₃): δ = 2.62(s, 3H), 2.71(s, 3H), 2.78(s, 3H), 3.88(s, 3H), 3.93(s, 6H), 7.08(m, 3H), 7.22(d, 1H), 8.46(dd, 1H), 9.40(d, 1H). |
| 19 | 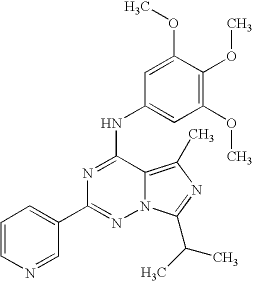 | LC/MS (method 6): Rₜ = 4.00 min MS (EI): m/z = 435 (M + H)⁺¹H-NMR (200 MHz, CDCl₃): δ = 1.47(d, 6H), 2.82(s, 3H), 3.70(quint. 1H), 3.89(s, 3H), 3.94(s, 6H), 7.04-7.16(m, 3H), 7.38(dd, 1H), 8.57(dt, 1H), 8.69(dd, 1H), 9.54(d, 1H). |

### Example 20

### 5,7-Dimethyl-2-(1-oxido-3-pyridinyl)-4-(3,4,5-trimethoxyphenoxy)imidazo[5,1-f]-[1,2,4]triazine

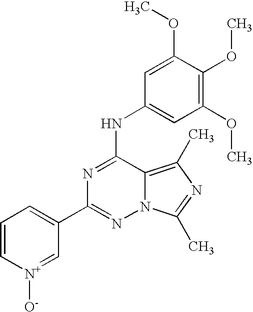

A solution of 55 mg (0.13 mmol) of 5,7-dimethyl-2-(3-pyridinyl)-4-(3,4,5-trimethoxyphenoxy)imidazo[5,1-f][1,2,4]triazine from example 7 introduced into 3 ml of dichloromethane is treated with 39.94 mg (0.16 mmol) of 3-chloroperbenzoic acid. In order to complete the reaction, after 3 hours a further 0.5 eq. of 3-chloroperbenzoic acid is added. After 30 minutes, the mixture is diluted with dichloromethane and washed with saturated aqueous sodium hydrogencarbonate solution. The organic phase is dried over magnesium sulfate, filtered and the solvent is removed under reduced pressure. The residue is purified by means of preparative HPLC.

Yield: 36 mg (63% of th.)

LC/MS (method 7): Rₜ=2.25 min

MS (EI): m/z=424 (M+H)⁺

¹H-NMR (300 MHz, CDCl₃): δ=2.74 (s, 3H), 2.75 (s, 3H), 3.87 (s, 6H), 3.92 (s, 3H), 6.53 (s, 2H), 7.30 (dd, 1H), 7.95 (dt, 1H), 8.24 (m, 1H), 8.99 (m, 1H).

### Example 21

### 7-Isopropyl-5-methyl-2-(1-oxido-3-pyridinyl)-4-(3,4,5-trimethoxyphenoxy)imidazo-[5,1-f][1,2,4]triazine

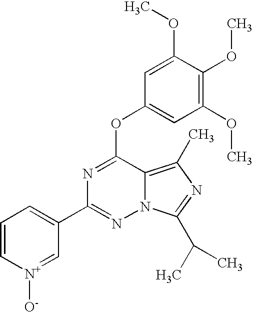

Preparation analogously to example 20 using 40 mg (0.09 mmol) of 7-isopropyl-5-methyl-2-(3-pyridinyl)-4-(3,4,5-trimethoxyphenoxy)imidazo[5,1-f][1,2,4]triazine from example 10, 3 ml of dichloromethane and 27.17 mg (0.11 mmol) and 11.32 mg (0.05 mmol) of 3-chloroperbenzoic acid.

Yield: 25 mg (60% of th.)

LC/MS (method 7): Rₜ=2.63 min

MS (EI): m/z=452 (M+H)⁺

¹H-NMR (300 MHz, CDCl₃): δ=1.47 (d, 6H), 2.74 (s, 3H), 3.67 (quint. 1H), 3.87 (s, 6H), 3.92 (s, 3H), 6.53 (s, 2H), 7.27-7.34 (m, 1H), 7.93 (d, 1H), 8.23 (d, 1H), 8.99 (s, 1H).

Preparation Analogously to Example 20:

| Example | Structure | Analytical data |
|---|---|---|
| 22 | 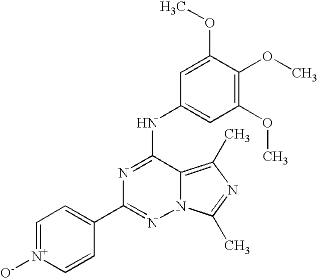 | LC/MS (method 7): Rₜ = 2.04 min MS (EI): m/z = 423 (M + H)⁺¹H-NMR (400 MHz, CDCl₃): δ = 2.78(s, 3H), 2.87(s, 3H), 3.90(s, 3H), 3.91(s, 6H), 7.00(s, 2H), 7.43(br. s, 1H), 8.22(m, 4H). |
| 23 | 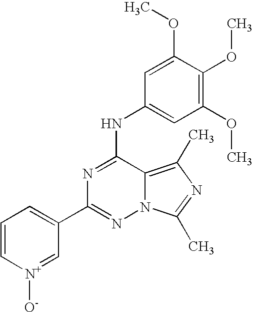 | LC/MS (method 7): Rₜ = 1.88 min MS (EI): m/z = 423 (M + H)⁺¹H-NMR (300 MHz, CDCl₃): δ = 2.69(s, 3H), 2.78(s, 3H), 3.89(s, 3H), 3.95(s, 6H), 7.00(s, 2H), 7.14(br. s, 1H), 7.29-7.37(m, 1H), 8.15(d, 1H), 8.25(d, 1H), 9.16(s, 1H). |

### Example 24

### 2-(6-Methoxy-3-pyridinyl)-5,7-dimethyl-N-(3,4,5-trimethoxyphenyl)imidazo[5,1-f]-[1,2,4]triazin-4-amine

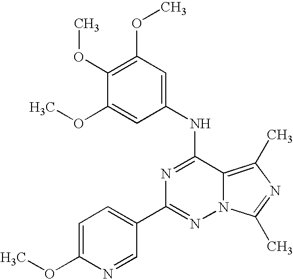

Preparation analogously to example 12 from 45 mg (0.25 mmol) of 3,4,5-trimethoxyaniline, 34 mg (0.25 mmol) of potassium carbonate and 40 mg (0.12 mmol) of 2-(6-methoxy-3-pyridinyl)-5,7-dimethyl-4-(1H-1,2,4-triazol-1-yl)imidazo[5,1-f]-[1,2,4]triazine from example 40A in 2 ml of DMF at 80° C. Subsequently, the crude solution is separated directly by means of HPLC.

Yield: 37 mg (68% of th.)

LC/MS (method 3): Rₜ=3.24 min

MS (ESI): m/z=438 (M+H)⁺

¹H-NMR (300 MHz, CDCl₃): δ=2.72 (s, 3H), 2.80 (s, 3H), 3.89 (3H), 3.94 (6H), 4.00 (s, 3H), 6.79 (d, 1H), 7.05-7.11 (m, 3H), 8.45 (dd, 1H), 9.12 (d, 1H).

### Example 25

### 2-(6-Methoxy-3-pyridinyl)-5,7-dimethyl-4-(3,4,5-trimethoxyphenoxy)imidazo[5,1-f]-[1,2,4]triazine

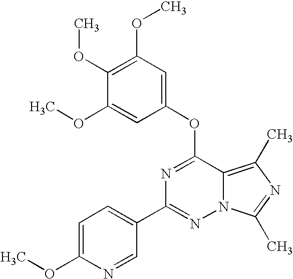

Preparation analogously to example 1 from 46 mg (0.25 mmol) of 3,4,5-trimethoxyphenol, 28 mg (0.25 mmol) of potassium tert-butoxide and 40 mg (0.12 mmol) of 2-(6-methoxy-3-pyridinyl)-5,7-dimethyl-4-(1H-1,2,4-triazol-1-yl)-imidazo[5,1-f][1,2,4]triazine from example 40A in 4 ml of tetrahydrofuran.

Yield: 24 mg (44% of th.)

LC/MS (method 3): Rₜ=2.70 min

MS (EI): m/z=437 (M+H)⁺

¹H-NMR (300 MHz, CDCl₃): δ=2.73 (s, 3H), 2.75 (s, 3H), 3.87 (s, 6H), 3.92 (s, 3H), 3.98 (s, 3H), 6.57 (s, 2H), 6.77 (d, 1H), 8.33 (dd, 1H), 8.90 (d, 1H).

### Example 26

### 2-(6-Chloro-3-pyridinyl)-5,7-dimethyl-N-(3,4,5-trimethoxyphenyl)imidazo[5,1-f]-[1,2,4]triazin-4-amine

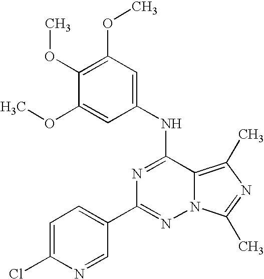

Preparation analogously to example 12 from 56 mg (0.31 mmol) of 3,4,5-trimethoxyaniline, 42 mg (0.31 mmol) of potassium carbonate and 50 mg (0.15 mmol) of 2-(6-chloro-3-pyridinyl)-5,7-dimethyl-4-(1H-1,2,4-triazol-1-yl)imidazo[5,1-f]-[1,2,4]triazine from example 38A in 2 ml DMF at 80° C. Subsequently, the crude solution is separated directly by means of HPLC.

Yield: 42 mg (62% of th.)

LC/MS (method 3): Rₜ=2.92 min

MS (ESI): m/z=441 (M+H)⁺

¹H-NMR (200 MHz, CDCl₃): δ=2.71 (s, 3H), 2.80 (s, 3H), 3.89 (s, 3H), 3.93 (s, 6H), 7.04 (s, 2H), 7.12 (br.s, 1H), 7.41 (d, 1H), 8.55 (dd, 1H), 9.30 (d, 1H).

### Example 27

### 5,7-Dimethyl-2-[6-(4-morpholinyl)-3-pyridinyl]-N-(3,4,5-trimethoxyphenyl)imidazo[5,1-f][1,2,4]triazin-4-amine

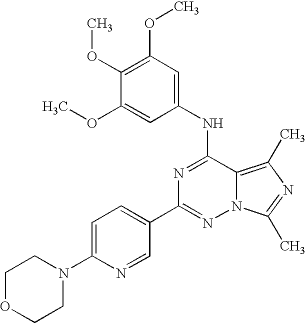

A mixture of 2 ml of morpholine, 20 mg (0.05 mmol) of 2-(6-chloro-3-pyridinyl)-5,7-dimethyl-N-(3,4,5-trimethoxyphenyl)imidazo[5,1-f][1,2,4]triazin-4-amine from example 26 and 13 mg (0.10 mmol) of potassium carbonate is heated overnight at 135° C. After cooling, the reaction mixture is treated with 15 ml of water and extracted three times with 15 ml each of dichloromethane. The organic phase is dried over magnesium sulfate and then freed from the solvent in vacuo. The residue is purified by means of HPLC.

Yield: 7.4 mg (33% of th.)

LC/MS (method 3): Rₜ=2.27 min

MS (ESI): m/z=492 (M+H)⁺

¹H-NMR (200 MHz, CDCl₃): δ=2.68 (s, 3H), 2.77 (s, 3H), 3.57-3.67 (m, 4H), 3.80-3.90 (m, 4H), 3.89 (s, 3H), 3.94 (s, 6H), 6.65 (d, 1H), 7.03 (br.s, 1H), 7.09 (s, 2H), 8.38 (dd, 1H), 9.15 (d, 1H).

1. A compound of the formula 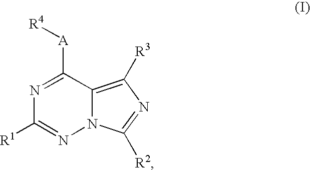
in which
R¹ denotes 5- to 10-membered heteroaryl, which can be substituted by up to 3 substituents selected independently of one another from the group consisting of oxo, halogen, carbamoyl, cyano, hydroxyl, (C₁-C₆-alkyl)carbonyl, trifluoromethyl, trifluoromethoxy, nitro, C₁-C₆-alkyl, C₁-C₆-alkoxy and —NR⁵R⁶,
where
R⁵ and R⁶ independently of one another denote for C₁-C₆-alkyl or
R⁵ and R⁶, together with the nitrogen atom to which they are bonded, denote a 5 to 8-membered heterocycle, which is optionally substituted by C₁-C₆-alkyl or C₁-C₆-hydroxyalkyl,
R² denotes C₁-C₆-alkyl or C₃-C₄-cycloalkyl,
R³ denotes methyl,
A denotes oxygen or NH, and
R⁴ denotes C₆-C₁₀-aryl, which can be substituted by up to 3 substituents selected independently of one another from the group consisting of halogen, formyl, carboxyl, carbamoyl, cyano, hydroxyl, trifluoro-methyl, trifluoromethoxy, nitro, C₁-C₆-alkyl, C₁-C₆-alkoxy, 1,3-dioxa-propane-1,3-diyl, C₁-C₆-alkylthio and —NR⁷R⁸,
in which
R⁷ and R⁸ independently of one another denote hydrogen, C₁-C₆-alkyl or C₁-C₆-alkylcarbonyl,
and their salts, solvates and/or solvates of the salts.

2. A compound as claimed in claim 1, where
R¹ denotes 5- to 10-membered heteroaryl, which can be substituted by up to 3 substituents selected independently of one another from the group consisting of oxo, C₁-C₆-alkyl, C₁-C₆-alkoxy and —NR⁵R⁶,
where
R⁵ and R⁶ independently of one another denote C₁-C₆-alkyl or
R⁵ and R⁶, together with the nitrogen atom to which they are bonded, form a 5 to 8-membered heterocycle, which is optionally substituted by C₁-C₆-alkyl or C₁-C₆-hydroxyalkyl,
R² denotes C₁-C₆-alkyl,
R³ denotes methyl,
A denotes oxygen or NH, and
R⁴ denotes phenyl, which can be substituted by up to 3 substituents selected independently of one another from the group consisting of halogen, C₁-C₆-alkyl and C₁-C₆-alkoxy,
and their salts, solvates and/or solvates of the salts.

3. A compound as claimed in claim 1 and 2, where
R¹ denotes thienyl, furyl, thiazolyl or pyridyl, which in each case can be substituted by up to 2 substituents selected independently of one another from the group consisting of oxo, C₁-C₆-alkyl, C₁-C₆-alkoxy and —NR⁵R⁶,
where
R⁵ and R⁶ independently of one another denote C₁-C₆-alkyl or
R⁵ and R⁶, together with the nitrogen atom to which they are bonded, form a 5 to 8-membered heterocycle, which is optionally substituted by C₁-C₆-alkyl or C₁-C₆-hydroxyalkyl,
R² denotes C₁-C₆-alkyl,
R³ denotes methyl,
A denotes oxygen or NH,
R⁴ denotes phenyl, which is substituted by up to 3 C₁-C₆-alkoxy radicals,
and their salts, solvates and/or solvates of the salts.

4. A process for the preparation of compounds as claimed in claim 1, characterized in that compounds of the general formula 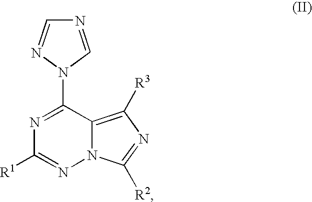
in which
R¹, R² and R³ have the meanings indicated in claim 1,
are reacted with compounds of the formula 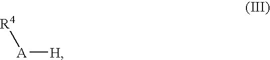
in which
R⁴ and A have the meanings indicated in claim 1,
to give compounds of the formula (I) and these are optionally reacted with the appropriate (i) solvents and/or (ii) bases or acids to give their solvates, salts and/or solvates of the salts.

5. (canceled)

6. A medicament containing at least one of the compounds as claimed in claim 1 and at least one pharmaceutically tolerable, essentially nontoxic vehicle or excipient.

7. A method for the treatment and/or prophylaxis of neurodegenerative disorders comprising administering to a human or animal an effective amount of a compound of claim 1 or a medicament of claim 6.

8. A method for the treatment and/or prophylaxis of cancer, neurodegenerative disorders or psychiatric disorders comprising administering to a human or animal an effective amount of a compound of claim 1 or a medicament of claim 6.

9. The method as claimed in claim 7, wherein the neurodegenerative disorder is Parkinson's disease.

10. The method as claimed in claim 8, wherein the psychiatric disorder is schizophrenia.

11. A process for the control of cancer, neurodegenerative disorders or psychiatric disorders in a human or animal comprising administering to a human or animal an effective amount of a compound of claim 1 or a medicament of claim 6.

12. The process as claimed in claim 11, wherein the neurodegenerative disorder is Parkinson's disease.

13. The process as claimed in claim 11, wherein the psychiatric disorder is schizophrenia.
